(19) United States
(12) Patent Application Publication
(10) Pub. No.: US 2011/0104078 A1
(43) Pub. Date: May 5, 2011

(54) UV ABSORBING COMPLEX POLYESTER POLYMERS, COMPOSITIONS CONTAINING UV ABSORBING COMPLEX POLYESTER POLYMERS, AND RELATED METHODS

(75) Inventors: Rocco Burgo, (US); Daniel Winn, (US)

(73) Assignee: Inolex Investment Corporation

(21) Appl. No.: 12/938,246

(22) Filed: Nov. 2, 2010

Related U.S. Application Data
(60) Provisional application No. 61/257,294, filed on Nov. 2, 2009.

(51) Int. Cl. A61K 8/85; C07D 413/14; C07C 69/94; A61Q 17/04; A61P 29/00; A61P 31/04; A61P 31/10; A61P 3/02; A61Q 19/04; A61Q 19/00; A61Q 19/02

(52) U.S. Cl. 424/43; 424/60; 548/220; 560/57

(57) ABSTRACT

The invention includes an UV absorbing complex polyol polyester polymer that is the product of a reaction scheme that includes: (i) the esterification of a polyol and a dianhydride, wherein the esterification is carried out under conditions that facilitate substantially only anhydride opening, to form a polyester polymer comprising at least two pendant carboxylic groups, and at least two hydroxyl groups; and (ii) the reaction of at least one pendant carboxylic group and at least one terminal hydroxyl group of the polyester polymer with an epoxide having a functional group, wherein the epoxide comprises an UV absorbing moiety.

Also included are linear UV absorbing complex polyol polyester polymers represented by Formula (XI):

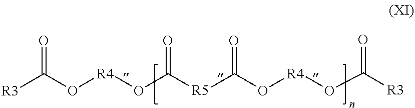

wherein R³ is independently selected from an UV absorbing moiety; R⁴ and R⁵ are each independently selected from a hydrocarbon group, and n is an integer of 1 to 1000.

A crosslinked UV absorbing complex polyol polyester polymer that is reaction product of a random copolyesterification esterification reaction and/or the esterification product of: a monofunctional carboxylic acid and/or ester that comprises an UV absorbing moiety, at least one of a diol, a polyol, a diacid and/or an ester is also included within the scope of the invention. The resulting polymer has an UV absorbing functionality of greater than 2.0.

### CROSS-REFERENCE TO RELATED APPLICATION

This application claims priority under 35 U.S.C. §119(e) to U.S. Provisional patent application 61/257,294, filed Nov. 2, 2009, the entire disclosure of which is incorporated herein by reference.

### BACKGROUND OF THE INVENTION

The electromagnetic radiation (light energy) within the ultraviolet (UV) spectrum that reaches the earth's surface falls within the wavelength range of approximately 290 to 400 nanometers (nm). The portion of the spectrum that is responsible for erythema (sunburn) of skin is within the range of about 290 to 320 nm, and is referred to as UV-B. More recently, research has shown that not only sunlight energy within the UV-B range can be harmful to skin, but lower energy, longer wavelengths (known as UV-A) with a range of 320 to 400 nm may also be problematic.

UV-A has been shown to penetrate the skin more deeply than UV-B. In studies which have occurred over the past two decades, it has been shown that the effects of prolonged UV-A exposure can result in premature skin aging, wrinkling, and has been implicated as a potential initiator for the development of skin cancers. UV-A damages skin cells in the basal layer of the epidermis (keratinocytes) where most skin cancers occur.

Topical photoprotective treatments, such as sunscreens, have been developed to mitigate or prevent skin damage. Sunscreen formulations are applied topically to protect against UV induced skin damage and are prepared in various forms, including creams, lotions, and sprays. Conventional sunscreen formulators will typically incorporate organic chemical compounds that chemically absorb UV radiation (organic UV filters) and inorganic compounds that in addition to absorbing, also physically scatter and/or reflect the radiation (UV blockers) into the sunscreen product.

For sunscreens to be used effectively, they need to be applied evenly and as directed. Misuse of sunscreens by improper or inconsistent application may result in a grave problem. The user may feel he or she is protected from the sun's rays and may take lesser steps to avoid exposure by physically covering the body by clothing or shade. Misapplication or under application can sometimes result because the user may feel that the sunscreen product is aesthetically unpleasing. Some UV filters, most notably those within the salicylate family such as 3,3,5-trimethylcyclohexyl 2-hydroxybenzoate (homosalate) and 2-ethylhexyl salicylate (octisalate) are somewhat viscous esters that impart an oily and/or greasy feel to the skin when the sunscreen product is applied. They also impart an odor to the sunscreen that many characterize as unpleasant. Due to the limited number of approved UV filters in the United States, the sunscreen formulator tends to utilize salicylates to achieve higher SPF products, despite these drawbacks. The user may tend to apply less than the recommended amount of the salicylate-containing sunscreen product because of the drawbacks, and may therefore receive lower levels of protection.

Historically, sunscreens were formulated predominantly to prevent sunburn and associated acute discomfort. Consequently, they included primarily UV-B filters and UV blockers. The ability of a given sunscreen to protect against sunburn is communicated to a consumer by use of the sun protection factor (“SPF”) system. SPF is an in-vivo laboratory measure of the effectiveness of sunscreen in preventing sunburn. It is a numerical value. The higher the SPF, the more protection a sunscreen offers against UV-B. The SPF is further defined, and the detailed testing procedures are provided in United States Food and Drug Administration (“FDA”) publication “Sunscreen Drug Products for Over-the-Counter Human Use; Final Monograph; 21CFR Parts 310, 352, 700 and 740. Federal Register 64 (98) May 21, 1999. pp. 27666-27693,” the contents of which are incorporated herein by reference. Hereinafter, this method of evaluating SPF shall be referred to as the “FDA SPF Method”.

Attempts have been made to develop sunscreens that include filters that also absorb UV-A radiation. To this point, the choice of unlimited approved organic UV-A filters in the United States is limited to butyl methoxydibenzoylmethane (avobenzone or AVO) due to statutory requirements. AVO has been shown to degrade in the presence of sunlight by photolytic mechanisms, with the products of photodegradation being less effective at absorbing UV-A radiation than the parent compound. This means that protection against UV-A is reduced from time of initial application and to upon subsequent exposure to sunlight when AVO is used as an UV-A filter. Photodegradation is particularly pronounced when AVO is used in combination with 2-ethylhexyl (2E)-3-(4-methoxyphenyl)prop-2-enoate (octylmethoxycinnamate, octinoxate, OMC.)

Regulatory activity has centered around the labeling of sunscreens and the development of better ways to convey to consumers a sunscreen's ability to not only protect against sunburn, but to also protect against UV-A damage. In 2007 the FDA published proposed amendments to the monograph for sunscreen drug products for over-the-counter human use. Within the amendments are revisions to the test-procedures for evaluating the efficacy of sunscreen products. In addition to SPF, the revisions include provisions for evaluating UV-A protection, as well as photostability. The FDA has also proposed a four-star UV-A protection rating system based on in-vivo and in-vitro testing methods. These values are further defined, and the detailed testing procedures are provided in, “U.S. Food and Drug Administration. Sunscreen Drug Products for Over-the-Counter Human Use; Proposed Amendment of Final Monograph; Proposed Rule; 21CFR Parts 347 and 352. Federal Register 72 (165) Aug. 27, 2007. 49070-4912”, the contents of which are incorporated herein by reference. Hereinafter, this method of evaluating UV-A protection shall be refers to as the “FDA Star Method”.

The European Cosmetics Association (“COLIPA”) has also published guidelines and testing procedures relating to UV-A protection. In these documents, additional numerical parameters have been defined such as the in-vitro SPF (SPF in vitro), and the in-vitro UV-A protection factor (UVAPF.) The “SPF in vitro” is defined by COLIPA as “the absolute protection performance of a sun care product against erythema-inducing radiation, calculated from the measured in-vitro transmittance and weighted with the erythema action spectrum.” The UVAPF is defined as “the absolute protection performances of a sun care product against UVA radiation calculated from the measured in-vitro transmittance after irradiation and weighted with the persistent pigment darkening (PPD) action spectrum.” These parameters are further defined and the detailed testing procedures are provided in “Colipa Project Team IV, in-vitro Photoprotection Methods, Method for the in-vitro Determination of UVA Protection Provided by Sunscreen Products, Guideline, 2007”, the contents of which are incorporated herein by reference. Hereinafter, this method of evaluating UV-A protection shall be referred to as the “COLIPA Guidelines.”

Additional parameters have been defined, such as the UV-A/UV-B ratio, and the critical wavelength. The UV-A/UV-B ratio describes the performance of a sunscreen in the UV-A in relation to its performance in the UV-B range. It is calculated as the ratio between the areas under the UV-A and UV-B parts of the extinction curve, both areas being normalized to the range of wavelengths involved. The UV-A/UV-B ratio is further defined and detailed testing procedures are provided in “Measurement of UV-A/UV-B ratio according to the Boots Star rating system (2008 revision.) Boots UK Limited, Nottingham, NG2 3AA, UK. January 2008”, the contents of which are incorporated herein by reference. Hereinafter, this method of determining the UV-A/UV-B ratio shall be referred to as the “Boots Method.”

The critical wavelength is given as the upper limit of the spectral range from 290 nm on, within which 90% of the area under the extinction curve of the whole UV-range between 290 nm and 400 nm is covered. If that wavelength is 370 nm or greater, the product is considered “broad spectrum,” which denotes balanced protection throughout the UV-B and UV-A ranges. The critical wavelength is further defined and detailed testing procedures are provided in “Diffey B L, Tanner P R, Matts P J, Nash J F. In-vitro assessment of the broad-spectrum ultraviolet protection of sunscreen products. J Amer Acad Dermatol 43:1024-35, 2000,” the contents of which are incorporated herein by reference and shall be referred to herein as the “Diffey Protocol.”

It has been discovered that certain sunscreen chemicals are absorbed across the skin and inter into systemic circulation. Particular attention has been given to the filter benzophenone-3 (“BP3”) as outlined in Benson H, Sarveiya C, Risk S, Roberts M. Influence of anatomical site and topical formulation on skin penetration of sunscreens. Clin Risk Manag. 2005 September; 1(3): 209-218, but can also be potentially attributed to other filters which tend to be low in molecular weight.

Thus, most sunscreen formulators aspire to develop a sun care product that, when tested, obtains higher values for some or all of the numerical parameters described above, and thereby achieve an improvement over current sunscreen technology, and which includes polymeric filters to mitigate skin penetration. There remains a need in the art for new ingredients, preferably polymers, that can be used to formulate photoprotective products such that improvements such as greater photostability, pleasant aesthetics, higher SPF, and increased UVA protection may be realized.

### BRIEF SUMMARY OF THE INVENTION

The invention includes an UV absorbing complex polyol polyester polymer that is the product of a reaction scheme that includes: (i) the esterification of a polyol and a dianhydride, wherein the esterification is carried out under conditions that facilitate substantially only anhydride opening, to form a polyester polymer comprising at least two pendant carboxylic groups, and at least two hydroxyl groups; and (ii) the reaction of at least one pendant carboxylic group and at least one terminal hydroxyl group of the polyester polymer with an epoxide having a functional group, wherein the epoxide comprises an UV absorbing moiety. In some embodiments, the polyol is a diol, and the dianhydride is UV absorbing and comprises a benzophenone moiety, wherein the esterification step of (i) yields a polyester polymer comprising a pendent carboxylic acid and a terminal hydroxyl group as represented by Formula (IX):

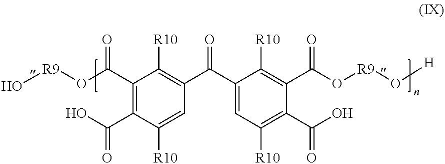

wherein R⁹ is independently selected from a hydrocarbon group having 2 to 54 carbon atoms, and 0 to 30 ether linkages, R¹⁰ is independently —H, or —OH, and n is an integer of 1 to 1000 or the dianhydride is not UV absorbing, and the esterification step of (i) yields a polyester polymer comprising at least two pendant carboxylic acid groups and two terminal hydroxyl groups represented by Formula (X):

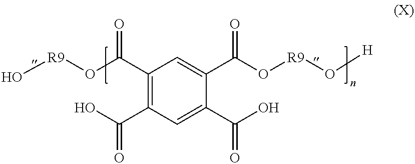

wherein R⁹ is independently selected from a hydrocarbon group having 2 to 54 carbon atoms, and 0 to 30 ether linkages, and n is an integer of 1 to 1000.

Also included are linear UV absorbing complex polyol polyester polymers represented by Formula (XI):

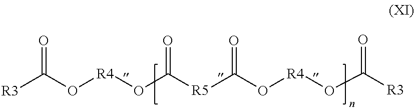

wherein R³ is independently selected from an UV absorbing moiety; R⁴ and R⁵ are each independently selected from a hydrocarbon group, and n is an integer of 1 to 1000.

A crosslinked UV absorbing complex polyol polyester polymer that is reaction product of a random copolyesterification esterification reaction and/or the esterification product of: a monofunctional carboxylic acid and/or ester that comprises an UV absorbing moiety, at least one of a diol, a polyol, a diacid and/or an ester is also included within the scope of the invention. The resulting polymer has an UV absorbing functionality of greater than 2.0.

Also included are crosslinked UV absorbing complex polyol polyester polymers that are the reaction product of a monofunctional agent comprising an UV absorbing moiety that has a structure represented by (XIII):

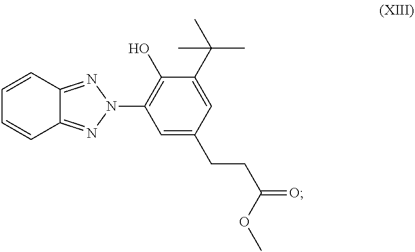

and additional reagents comprising those having the structures represented by (XIV) to (XV):

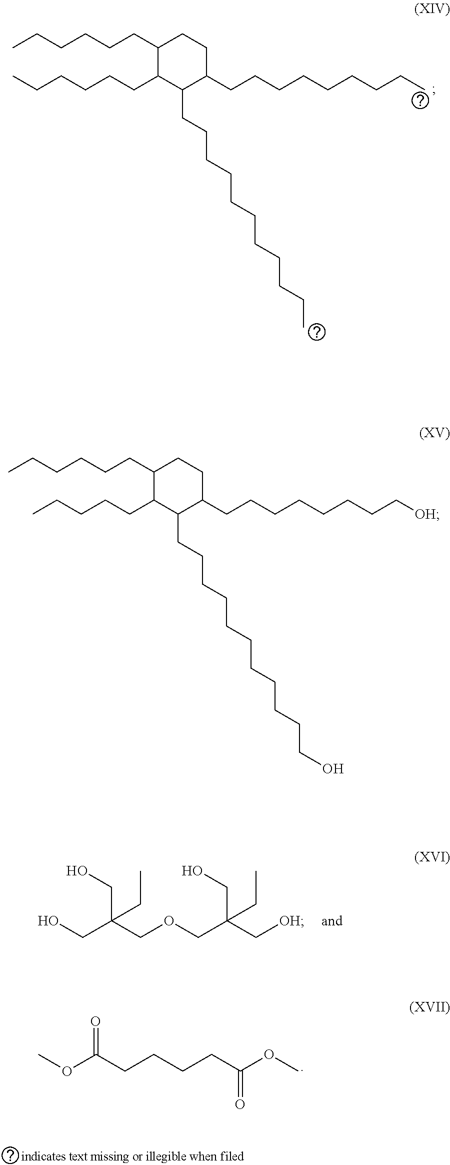

Also included are personal care compositions containing one or more polymers of the invention, and related methods, such as methods of increasing the photostability of the personal care compositions, methods of increasing the SPF or the UV-A protection provided by a photoprotective personal care composition, and/or methods of protecting the hair, skin or nails of a mammal using the compositions and polymers of the invention.

### BRIEF DESCRIPTION OF THE SEVERAL VIEWS OF THE DRAWINGS

The foregoing summary as well as the following detailed description of embodiments of the invention may be better understood when read in conjunction with the appended figures. It should be understood that the invention is not limited to the precise arrangements and instrumentalities shown. In the figures:

FIGS. 1A and 1B show the FTIR spectrum and the UV spectrum, respectively of the polymer described in Example 1;

FIGS. 2A, 2B, 2C, and 2D show the FTIR spectrum and the UV spectrum, respectively of the polymers described in Example 2;

FIG. 3A, 3B, and 3C shows the UV absorbance (A) as a function of wavelength during irradiation of each sample evaluated in Example 3;

FIG. 4 shows the UV spectrum for sunscreens evaluated in Example 4.

FIG. 5 shows the UV absorbance (A) as a function of wavelength for each blend evaluated in Example 6; and

FIG. 6 shows the UV absorbance (A) as a function of wavelength for each sample evaluated in Example 7.

### DETAILED DESCRIPTION OF THE INVENTION

The invention includes personal care compositions containing complex polyol polyester polymer compounds, and related methods. Also included are photostabilized personal care compositions wherein the addition of the UV absorbing complex polyol polyester polymers of the invention facilitates photostabilization of the photoprotective compositions that occur include other non-polymeric photoprotective ingredients. Synergistic compositions including mixtures of the complex polyester polymers of the invention with other photoprotective ingredients are also contemplated. It has been discovered that the addition of the complex polyol polyester polymers of the invention increases the SPF more than would be predicted using a model based upon the extinction coefficient of the base components. Methods to improve the aesthetics of photoprotective personal care compositions are also included as are other related methods.

The polymer of the invention includes a complex polyol polyester polymer. By “complex polyol polyester,” it is meant compounds that include a polyol polyester polymer backbone that is derived through esterification and/or transesterification reactions of polyols, polyacids, polyanhydrides and/or polyesters, that are fully or partially terminated by reaction with monofunctional acids, anhydrides, monofunctional alcohols, monofunctional epoxides and/or monofunctional esters. By “backbone,” it is meant a sequence of monomers comprising polyols, polyacids, polyanhydrides and/or polyesters linked together through ester linkages. “Polyanhydrides,” as used herein, are discrete chemical entities that contain two or more anhydride groups.

In the polymers of the invention, an UV-absorbing moiety is incorporated or linked into the structure of the complex polyol polyester polymer. This incorporation or linkage may occur by including the selected UV-absorbing moiety into one or more of the categories of initial reactants. Any variety (i.e., structure and molecular weight) of compounds that fall within the initial reactant categories may be used. Reactants categories include diols, polyacids, polyester, monofunctional alcohols, esters, acids and/or epoxides and the like.

Suitable diols may include branched and/or linear, saturated and/or unsaturated, aliphatic and/or aromatic containing two to fifty four carbon atoms and two to ten hydroxyl groups. Such polyols may omit any UV absorbing moiety or may contain an UV absorbing entity. Examples of preferred diols are without limitation, ethylene glycol 1,2-propanediol; 1,3-propanediol; 1,3-butylene glycol; 1,4-butanediol; 2-methyl-1,3-propanediol; diethylene glycol; tetraethylene glycol; 1,5-pentanediol; neopentyl glycol; 1,6-hexanediol; dipropylene glycol; 1,2-octanediol; and dimerdiol.

Exemplary polyacids are branched and/or linear, saturated and/or unsaturated, aliphatic and/or aromatic containing two to fifty four carbon atoms, two to four carboxylic acid and/or anhydride groups, up to zero to two sulfonic acid (and salts thereof) groups. Examples of preferred polyacids are without limitation, carbonic acid; propanedioic acid; decanedioic acid; pentanedioic acid; hexanedioic acid; heptanedioic acid; octanedioic acid; nonanedioic acid; decanedioic acid; dimer acid; trimer acid; tetramer acid; phthalic acid; isophthalic acid; pyromellitic acid; naphthylene dicarboxylic acid; and sodiosulfophthalic acid. Such polyacids may omit any UV-absorbing moiety or may contain an UV-absorbing entity.

Exemplary polyesters are those derived from any of the polyacids listed above, and/or further derived from at least one monofunctional alcohol comprising branched and/or linear, saturated and/or unsaturated, aliphatic and/or aromatic monofunctional alcohols containing one to thirty six carbon atoms. Examples of preferred monofunctional alcohols for the preparation of the polyesters are without limitation; methanol; ethanol; 1-butanol; isobutanol; 1-pentanol; 1-hexanol; 1-octanol; 2-ethyl-1-hexanol; 1-nonanol; and 1-decanol. Such polyesters may omit any UV-absorbing moiety or may contain an UV-absorbing entity.

Exemplary monofunctional alcohols that do not contain an UV absorbing moiety are branched and/or linear, saturated and/or unsaturated, aliphatic and/or aromatic monofunctional alcohols containing one to thirty six carbon atoms.

Exemplary monofunctional acids are branched and/or linear, saturated and/or unsaturated, aliphatic and/or aromatic containing one to thirty six carbon atoms. Such acids may omit any UV-absorbing moiety or may contain an UV-absorbing entity.

Exemplary monofunctional esters are branched and/or linear, saturated and/or unsaturated, aliphatic and/or aromatic containing one to thirty six carbon atoms. Such esters may omit any UV-absorbing moiety or may contain an UV-absorbing entity.

Exemplary monofunctional epoxides are branched and/or linear, saturated and/or unsaturated, aliphatic and/or aromatic containing one to thirty six carbon atoms. Such epoxides contain an UV-absorbing entity.

Various modifications in the selection and permutation of reactants, well within the skill set of a person of ordinary skill, may be made depending on the other selected reactants and/or to encourage the formation of a final product having a targeted property. For example, when forming the complex polyol polyester polymer that utilizes an epoxide in the synthesis, dianhydrides may be preferred. When forming a complex polyol polyester polymer with water soluble and/or dispersible properties, dianhydrides or sulfonic acid (and salts thereof) functional group containing diacids or anhydrides may be preferred. Particularly preferred polyacids that are utilized for the formation of a water soluble and/or water dispersible complex polyol polyester polymer may be sodiosulfophthalic acid and pyromellitic acid.

The UV absorbing moiety that is part of the structure of a reactant falling within one or more of the above categories may absorb predominantly in the UV-A or UV-B region of the spectrum. Alternatively, it may be a broad spectrum UV absorber.

In some embodiments, it may be preferred that the UV absorbing moiety is a derivatized benzophenone moiety, derivatized naphthalene moiety, and/or a benzotriazole derivative. Alternatively, the UV absorbing moiety may have the chemical structure of, or be similar to (i.e., be a derivative of) bis-ethylhexyloxyphenol methoxyphenyl triazine; butyl methoxydibenzoylmethane; diethylamino hydroxybenzoyl hexyl benzoate; disodium phenyl dibenzimidazole tetrasulfonate; drometrizole trisiloxane; methylene bis-benzotriazolyl tetramethylbutylphenol; terephthalylidene dicamphor sulfonic acid; menthyl anthranilate; methylene bis-benzotriazolyl tetramethylbutylphenol; 4-methylbenzylidene camphor; benzophenone-3; benzophenone-4; diethylhexyl butamido triazone; ethylhexyl methoxycinnamate; ethylhexyl salicylate; ethylhexyl triazone; ethylhexyl dimethyl PABA; homomethyl salicylate; isoamyl p-methoxycinnamate; octocrylene; phenylbenzimidazol sulfonic acid; polysilicone-15; benzotriazolyl dodecyl p-cresol; butyloctyl salicylate; diethylhexyl 2,6-naphthalate; diethylhexyl syringylidene malonate and polyester-8, so long at it is structurally incorporated into the polymer.

Examples of reactants that contain a benzotriazole group and which may be used in the preparation are provided by Formula (I):

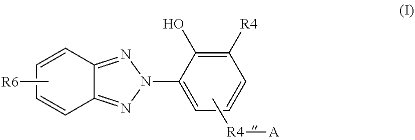

wherein R⁶ is independently a hydrogen atom or halogen atom, R⁴ is a substituted or unsubstituted hydrocarbon group, and A is a functional group selected from the group consisting of carboxylic acid, ester, and/or epoxide. Preferred may be of benzenepropanoic acid, 3-(2H-benzotriazol-2-yl)-5-(1,1-dimethylethyl)-4-hydroxy-, alkyl ester; benzenepropanoic acid, 3-(2H-benzotriazol-2-yl)-5-(1,1-dimethylethyl)-4-hydroxy-; benzenepropanoic acid, 3-(5-chloro-2H-benzotriazol-2-yl)-5-(1,1-dimethylethyl)-4-hydroxy-, alkyl ester and; 3-(5-chloro-2H-benzotriazol-2-yl)-5-(1,1-dimethylethyl)-4-hydroxy-, and/or derivatives thereof. When the A group is not present (e.g., it has been or will be reacted), this structure is referred to as Formula (Ia) as described herein.

In one embodiment, a dianhydride containing an UV absorbing moiety is esterified under conditions that substantially favor anhydride opening with one or more diols yielding a precursor linear hydroxyl terminated polyester polymer with pendant carboxylic acid groups. In a second step, the precursor polymer is further derivatized by reaction with an UV absorbing epoxide creating additional ester linkages, and ether linkages. An exemplary reaction scheme utilizing benzophenone tetracarboxylic acid dianhydride, one or more diols, and naphthyl glycidyl ether is depicted in Scheme 1.

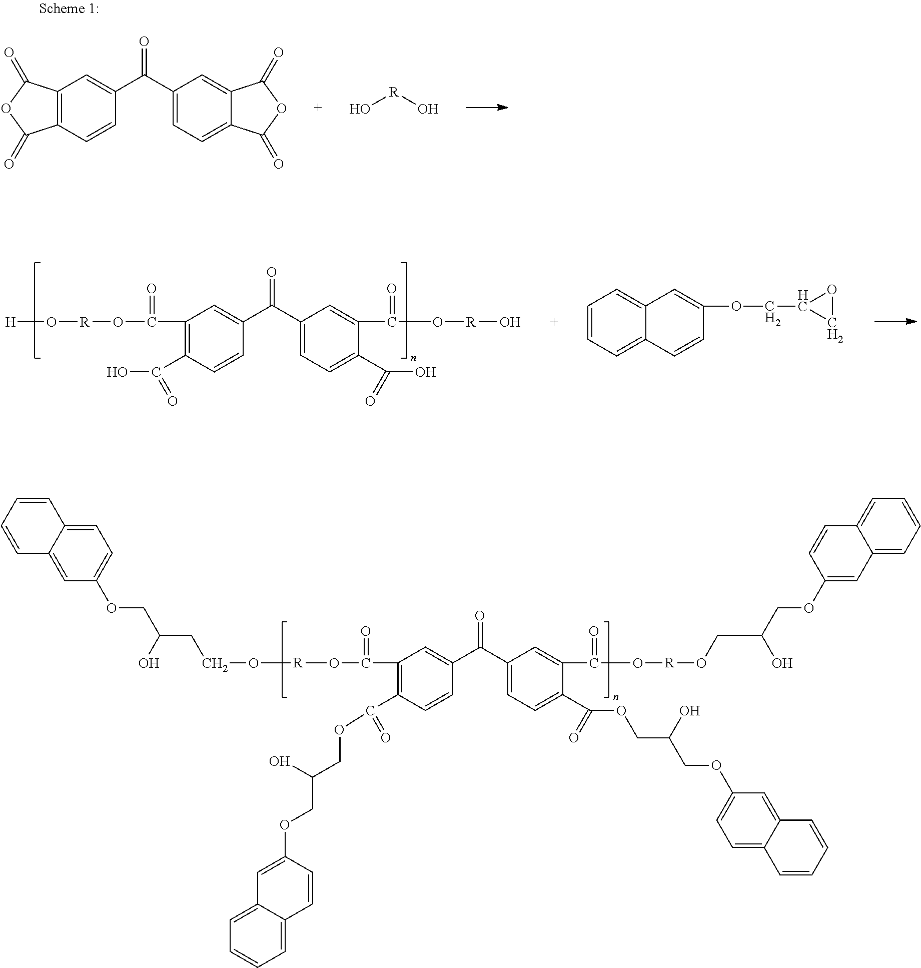

UV absorbing epoxides may typically prepared by the reaction of an UV absorbing alcohol or an UV absorbing carboxylic acid with an epihalohydrin followed by treatment with base. Any epihalohydrin may be used for the preparation of the UV absorbing epoxides of the invention. It may be preferred to use epichlorohydrin as it reacts conveniently and quantitatively with compounds bearing hydroxyl and/or carboxylic acid groups, which can be used as intermediates for the formation of the polymers of the invention. For example, Scheme 2 depicts the reaction of UV absorbing alcohol with epichlorohydrin to form a vicinal halohydrin, which is then converted back to the epoxide by treatment with base.

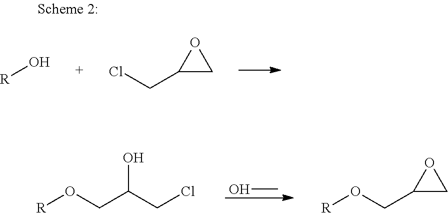

wherein R represents an UV absorbing moiety.

As another example, an epoxide bearing an UV absorbing moiety may be conveniently prepared from an alcohol bearing an UV absorbing moiety by this method as depicted in Scheme 3. In the example, the alcohol is 2-naphthol.

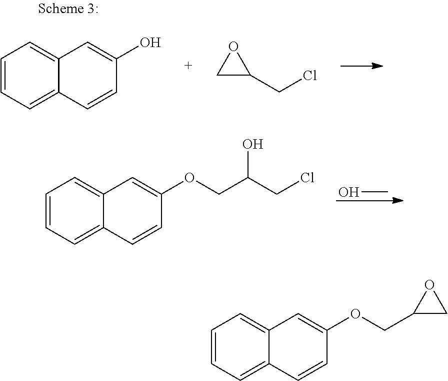

Also exemplary, Scheme 4 depicts the reaction of an UV absorbing carboxylic acid with epichlorohydrin to form a vicinal halohydrin, which is then converted back to the epoxide by treatment with base.

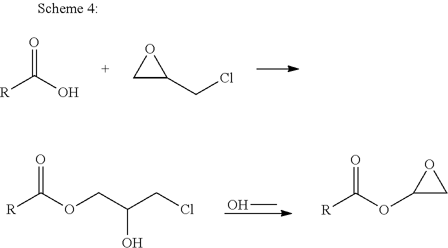

wherein R represents an UV absorbing moiety.

As an alternative example, an epoxide bearing an UV absorbing moiety may also be conveniently prepared from a carboxylic acid bearing an UV absorbing compound by this method as depicted in Scheme 5.

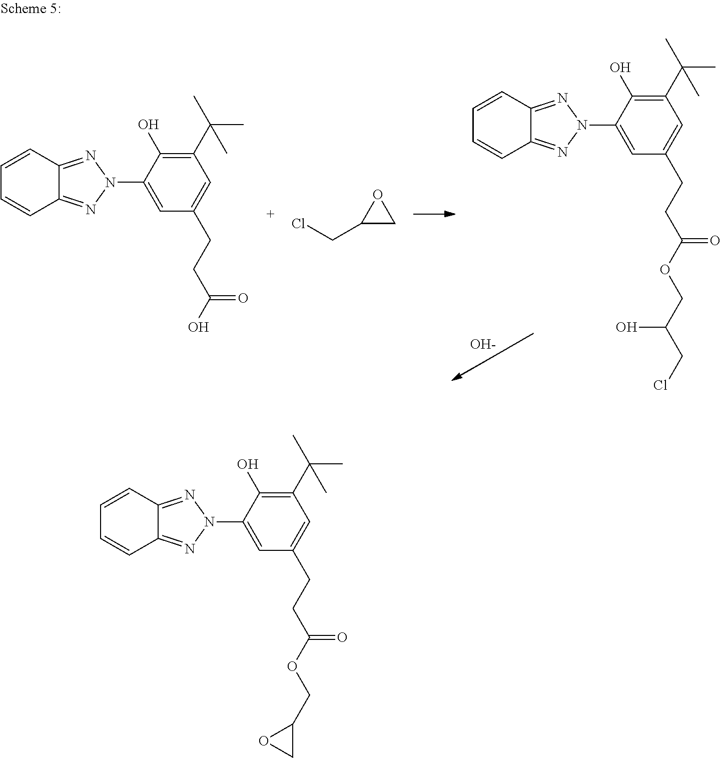

In the example of Scheme 5, the carboxylic acid is benzenepropanoic acid, 3-(2H-benzotriazol-2-yl)-5-(1,1-dimethylethyl)-4-hydroxy. Implementing Schemes 2 and/or 4 provides UV absorbing epoxides to form polymers suitable for inclusion in a personal care composition based on the chemistry represented in Scheme 1 from any alcohol or carboxylic acid that includes an UV absorbing moiety.

Exemplary UV absorbing alcohols that can be used to form UV absorbing epoxides are represented in Formulas (II), (III), (IV) and (V):

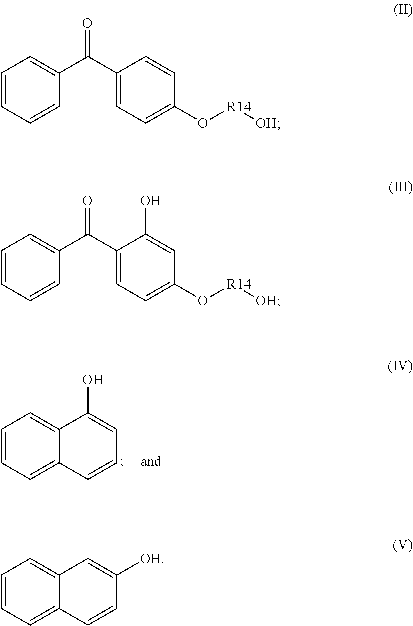

R¹⁴ in each instance may be independently any hydrocarbon group, including, for example, those that are substituted or unsubstituted, branched, unbranched and/or cyclic or ring structures and may contain, for example, 1 to 50 carbon atoms. Other examples may include methanone, [4-(2-hydroxyethoxy)phenyl]phenyl- and methanone, [2-hydroxy-4-(2-hydroxyethoxy)phenyl]phenyl-.

Exemplary UV absorbing carboxylic acids that can be used to form UV absorbing epoxides are represented in Formulas (VI) and (VII).

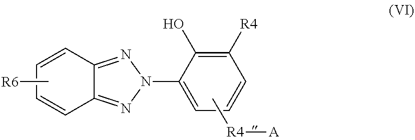

wherein R⁶ is independently an hydrogen atom or halogen atom, R⁴ is a hydrocarbon group, substituted to unsubstituted, and A¹ is a carboxylic acid group, and

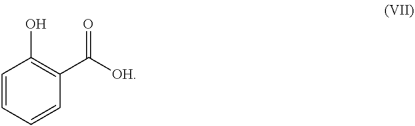

In another embodiment, a water soluble and/or dispersible UV absorbing acid functional polyol polyester polymer is prepared by esterification of a dianhydride containing an UV absorbing group by anhydride opening with one or more diols yielding a hydroxyl and carboxylic acid functional polyol polyester polymer. A portion of the hydroxyl and carboxylic acid groups are then etherified and/or esterified by an epoxide preferably containing an UV absorbing group. The remaining carboxylic acid groups are then neutralized with a base.

In another embodiment of the invention, a cross linked complex polyester polymer (crosspolymer) is formed that includes a benzotriazole group as the UV-absorbing moiety. By “cross linked,” it is meant herein that at least one of the polyfunctional monomers that contains only carboxylic acid (or ester) groups, or at least one of the polyfunctional monomers that contains only hydroxyl groups, or at least one of the polyfunctional monomers that contains both carboxylic acid (or ester) and hydroxyl groups, have at least three total functional groups, and are used in the formation of the polyester polymer backbone. By “cross link density,” it is meant herein as the number of cross link sites per mole of polymer. By “complex,” it is meant herein that the terminal carboxylic acid (or ester) and/or hydroxyl groups in the polymer backbone are “capped” with a monofunctional compound. By “capped,” it is meant herein that the terminal functional groups of the polyester polymer backbone are derivatized by monofunctional reactants. “UV absorbing moiety density” is herein defined as the number of moles of UV absorbing moiety on average divided by the total number of moles of polymer. For example, a benzotriazole group containing methyl ester can be co-transesterified with one or more diols and/or dimethyl esters with at least one polyol containing three or more hydroxyl groups resulting in a cross linked complex polyester polymer that has an UV absorbing moiety density that is greater than two. The reaction may be carried out in a single pot reaction. Optionally, but typically, a catalyst is employed. For example, Scheme 6 shows a reaction in accordance with the invention that involves the transesterification of three moles of benzenepropanoic acid, 3-(2H-benzotriazol-2-yl)-5-(1,1-dimethylethyl)-4-hydroxy, methyl ester with three moles mole of dimethyl ester, three moles of diol, and one mole of triol.

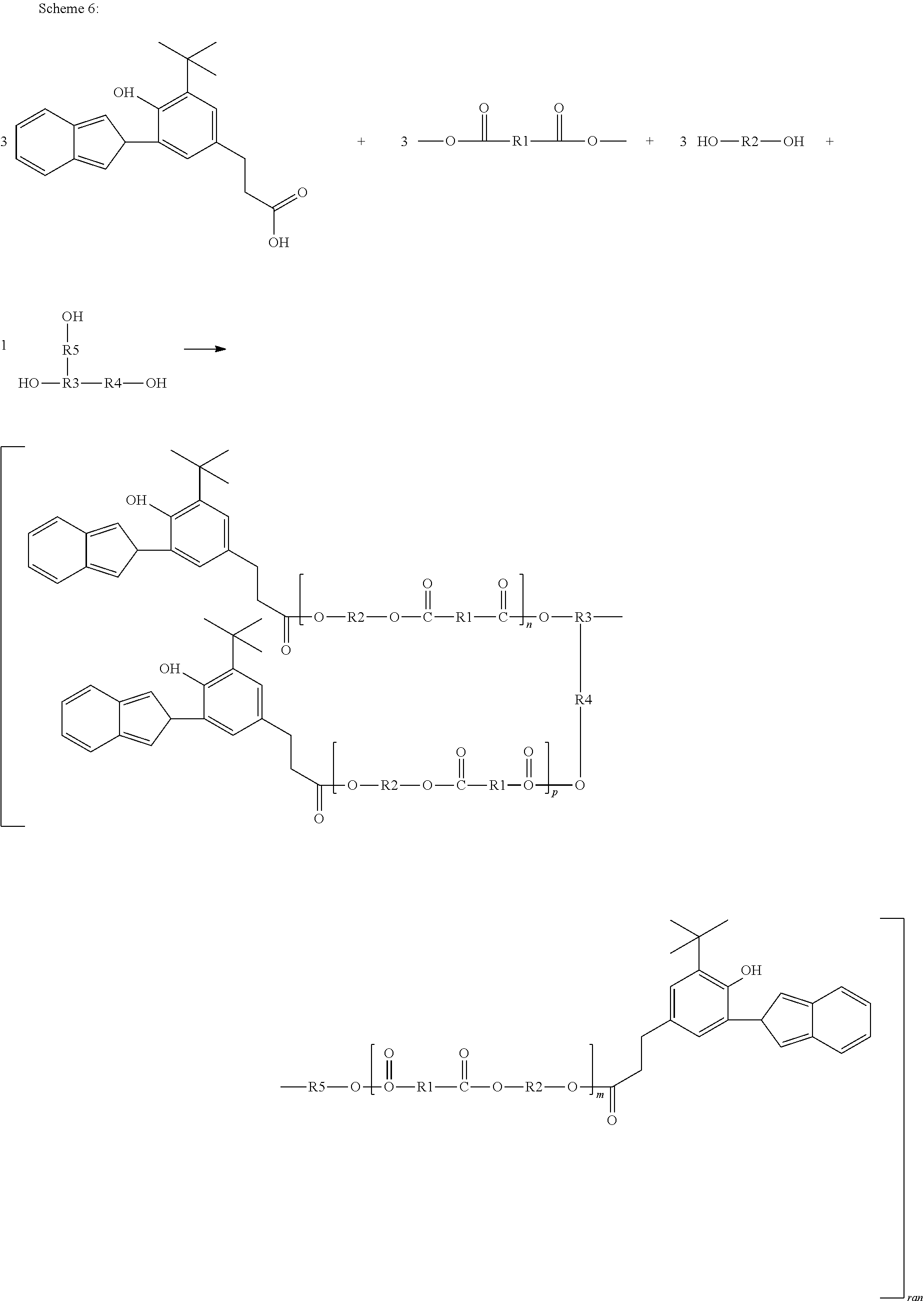

The structure of complex polyester polymer depicted in Scheme 6 represents an idealized structure. The resultant polymer, suitable for inclusion in a personal care composition may be cross linked with a cross link density equal to one, and an UV absorbing moiety density equal to three.

In another embodiment of the invention, a linear UV absorbing complex polyester polymer is formed that includes a benzotriazole group as the UV-absorbing moiety. A benzotriazole group containing methyl ester may be transesterified with one or more diols and/or diacid methyl esters in a single pot reaction as shown in the chemistry depicted in Scheme 7. Optionally a catalyst is employed.

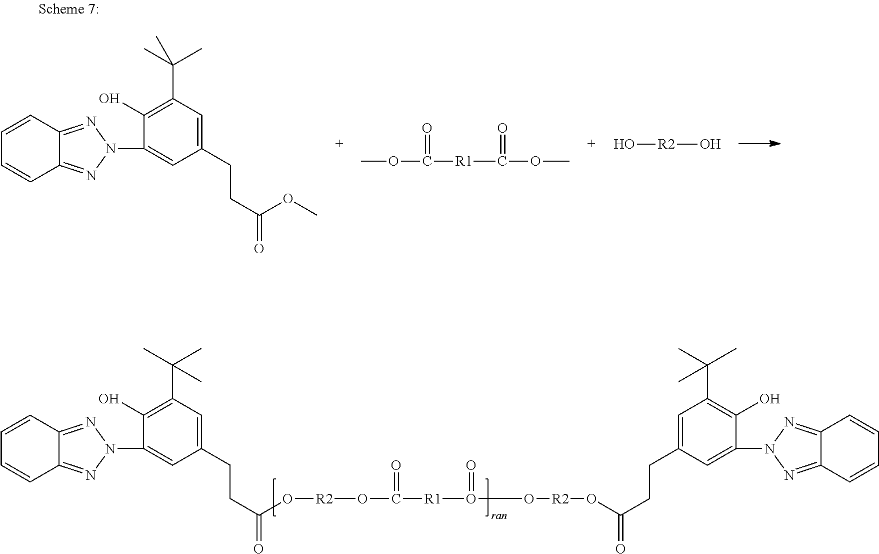

The resultant polymer, suitable for inclusion in a personal care composition is not cross linked (a cross link density equal to zero), and an UV absorbing moiety density equal to two.

Using the monomers, intermediate polymers and idealized reaction schemes described and articulated herein; one may derive numerous polymers of the invention. In an embodiment, one of these polymers is an UV absorbing complex polyol polyester polymer that is the product of a reaction scheme comprising: (i) the esterification of a polyol and a dianhydride, wherein the esterification is carried out under conditions that facilitate substantially only anhydride opening, to form a polyester polymer comprising at least two pendant carboxylic groups, and at least two hydroxyl groups; and (ii) the reaction of at least one pendant carboxylic group and at least one terminal hydroxyl group of the polyester polymer with an epoxide having a functional group, wherein the epoxide comprises an UV absorbing moiety.

By “UV absorbing,” as used herein, it is meant that the moiety absorbs radiation in the ultraviolet spectrum within the range of about 290 to about 400 nm. “Polyester polymer,” as used herein, refers to a polymer that is formed from the esterification and/or transesterification of monomer units of compounds containing two or more carboxylic acid groups and/or two or more ester groups and/or two or more hydroxyl groups, wherein the monomers are attached to one another by ester linkages. By “anhydride opening”, as used herein, it is meant a reaction between an anhydride and an alcohol thus forming an ester linkage and conditions that facilitate substantially only anhydride opening include those, well known in the art, under which 70% or greater anhydride opening occurs.

In an embodiment, the polyol may be preferred to be a diol and the anhydride may be UV absorbing or may not have the ability to absorb UV wavelengths (“not UV absorbing”). In addition, it may be preferred that the anhydride contains a benzophenone moiety.

In an embodiment, the esterification reaction described in (i) above may yield a polyester polymer comprising a pendent carboxylic acid and a terminal hydroxyl group as represented by Formula (IX):

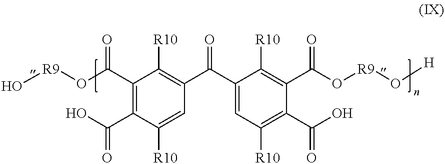

wherein R⁹ is independently selected from a hydrocarbon group, for example, having 2 to 54 carbon atoms and 0 to 30 ether linkages, R¹⁰ is independently —H, or —OH, and n is an integer of 1 to 1000.

Alternatively, the esterification reaction described in (i) above may yield a polyester polymer comprising at least two pendant carboxylic acid groups and two terminal hydroxyl groups represented by Formula (X):

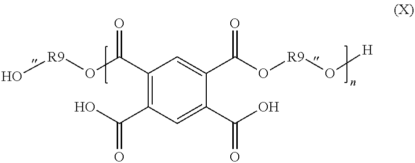

wherein R⁹ is independently selected from a hydrocarbon group having, for example, 2 to 54 carbon atoms, and 0 to 30 ether linkages, and n is an integer of 1 to 1000.

In each embodiment, the reaction of step (ii) comprises the etherification reaction of the functional group of the epoxide with at least one of the hydroxyl and/or carboxylic acid groups of the polyester polymer, and the polymer of the invention is formed.

As an example, in step (i), the esterification may be conducted between 3,3′,4,4′-benzophenone tetracarboxylic dianhydride (BTDA) and a diol under conditions where substantially only anhydride opening occurs. A precursor polyester polymer is formed; it contains a terminal hydroxyl groups, and pendant carboxylic acid groups. In a subsequent step (step (ii)), an epoxide containing an UV absorbing moiety is used to further derivatize the residual active hydrogen containing functional groups of the precursor polyester polymer by full or partial etherification of the terminal hydroxyl groups, and full or partial esterification of the pendant carboxylic acid groups.

In some embodiments, the epoxide may be derived from the epoxidation of an UV absorbing alcohol and/or of an UV absorbing carboxylic acid. The UV absorbing moiety of the epoxide is selected from a derivatized benzophenone moiety, derivatized naphthalene moiety, and a benzotriazole derivative.

Alternatively or additionally, the epoxide used may be derived from a reaction represented by the reaction schemes 8A and/or 8B:

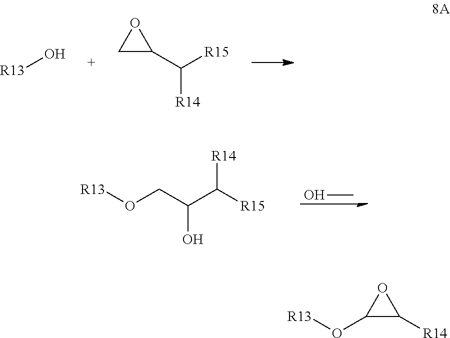

In this 8A, R¹³ comprises an UV absorbing moiety, and R¹⁴ is independently selected from a hydrogen atom, and a hydrocarbon group having, for example, 1 to 54 carbon atoms and 0 to 30 ether linkages, and R¹⁵ is an halogen atom; or

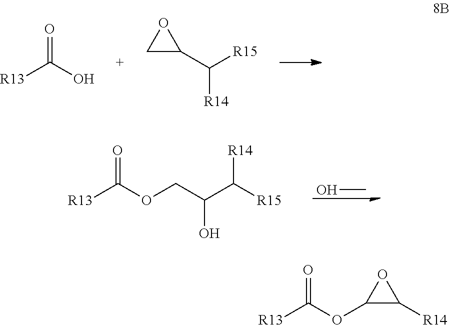

In 8B, R¹³ comprises an UV absorbing moiety, and R¹⁴ is independently selected from a hydrogen atom, and a hydrocarbon group having, for example, 1 to 54 carbon atoms and 0 to 30 ether linkages, and R¹⁵ is a halogen atom.

In an additional embodiment, the polymer is a linear UV absorbing complex polyol polyester polymer represented by Formula (XI):

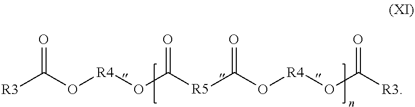

In XI, R³ is independently selected from an UV absorbing moiety; R⁴ and R⁵ are each independently selected from a hydrocarbon group, and n is an integer of 1 to 1000. By “linear” it is meant herein that the polymer backbone is formed by the linking of any categories of the reactants containing only two or less functional groups.

The UV absorbing moiety is chosen from a compound containing an UV absorbing benzotriazole group. In some circumstances, it may be preferred that the UV absorbing benzotriazole group is represented by the structure (Ia):

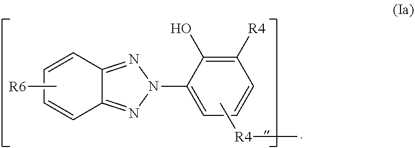

In Ia, R⁶ is independently a hydrogen atom or a halogen atom, and R⁴ is a hydrocarbon group. In some embodiments, R⁴ and R⁵ are each independently selected from a hydrocarbon group having, for example, 2 to 54 carbon atoms, and 0 to 30 ether linkages, wherein each of the carbons of the hydrocarbon group is independently substituted or unsubstituted, and saturated or unsaturated.

An example may include the polymer where R⁴ is a substituted or unsubstituted alkyl chain containing two to 36 carbon atoms and/or one to 400 ether linkages, and/or R⁵ is a substituted or unsubstituted alkyl chain containing two to 54 carbon atoms, and/or linear and/or branched, and/or aromatic, and/or cyclic, and or polycyclic, R³ is an UV absorbing residue comprising a substituted triazole, a substituted benzophenone, and/or a substituted naphthyl group, and n equals 0 to 1000. By “residue,” it is meant that an UV absorbing moiety is attached to a functional group that has the capability of reacting with the polyol polyester backbone in accordance with the invention.

Also included are polymers of the invention that are crosslinked, such as a crosslinked UV absorbing complex polyol polyester polymer that is reaction product of a random copolyesterification esterification reaction and/or the esterification product of: a monofunctional carboxylic acid and/or ester that comprises an UV absorbing moiety, at least one of a diol, a polyol, a diacid and/or an ester, wherein the polymer has an UV absorbing functionality of greater than 2.0. In some embodiments, the UV functionality may be about 3 to about 50, about 5 to about 25 and about 10 to about 20. By “random,” it is meant that the monomer reactants are linked together in no particular sequence, and will link together based upon the laws of probability and/or mass action. By “cross linked,” it is meant that least one of the categories of the reactants contains at least three functional groups.

In some embodiments, the monofunctional carboxylic acid and/or ester is represented by Formula (I):

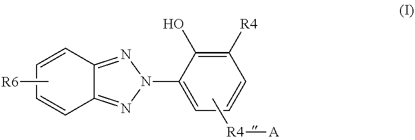

wherein R⁶ is independently selected from a hydrogen atom or a halogen atom, R⁴ is a hydrocarbon group, and A is a functional group selected from the group consisting of carboxylic acid and ester.

As noted, the polymers of the invention (and, in some cases the monomers and/or moieties that make up the polymers) are obtained by reaction of varied precursor molecules. For that reason, the hydrocarbon groups presented will necessarily vary, depending on the precursor molecules. Thus, the hydrocarbon groups described herein may be independently substituted or unsubstituted, functionalized or not functionalized, may be alkyl, aryl, alkene, alkyne, aklyne, may have branched or ring structures and may contain 1, 2, 3, 4, 5, 6, 7, 8, 9, 10 carbon atoms or 1 to 500 carbon atoms, 100 to 300 carbon atoms, and/or 10-55 carbons atoms.

Any of the above-described polymers may be incorporated into a personal care composition. In addition to the polymers, the composition may include any personal care ingredients known in the art, such as surfactants, buffers, perfumes, colorants, dyes, viscosity modifiers, water, oils, emulsifiers, preservatives, antioxidants, emollients, thickeners, gellants, vitamins, humectants, alcohols, botanical extracts and powders. Other suitable additive or components include may include one or more vegetable oils in the product, such as, for example, almond oil, castor oil, coconut oil, corn (maize) oil, cottonseed oil, canola oil, flax seed oil, hempseed oil, nut oil, olive oil, palm oil, peanut oil, safflower oil, sesame oil, soybean oil, sunflower oil, jojoba oil and combinations of these oils.

Surfactants may be included in the personal care composition, such as, for example, an anionic surfactant, a zwitterionic surfactant, a cationic surfactant, a non-ionic surfactant and combinations of these. Other exemplary components or additives may include, without limitation, lipids, alcohols, waxes, pigments, vitamins, fragrances, bleaching agents, antibacterial agents, anti-inflammatory agents, antimycotic agents, thickeners, gums, starches, chitosan, polymeric materials, cellulosic materials, glycerin, proteins, amino acids, keratin fibers, fatty acids, siloxanes, botanical extracts, abrasives and/or exfoliants (chemical or mechanical), anticaking agents, antioxidant agents, binders, biological additives, buffering agents, bulking agents, chelating agents, chemical additives, denaturants, external analgesics, film formers, humectants, opacifying agents, pH adjusters, preservatives, propellants, reducing agents, sunscreen agents, skin darkening agents, essential oils, skin sensates, and combinations of these.

In addition to the polymer of the invention, the personal care composition may also include at least one additional UV protective agent, such as non-polymeric chemical UV filters. Such agents or filters may include octyl triazone, diethylamino hydroxybenzoyl hexyl benzoate, iscotrizinol, dimethico-diethylbenzalmalonate, polysilicone-15, isopentenyl-4-methoxycinnamate, p-aminobenzoic acid, octyldimethyl-PABA, phenylbenzimidazole sulfonic acid, 2-ethoxyethyl p-methoxycinnamate, benzophenone-8, benzophenone-3, homomethyl salicylate, meradimate, octocrylene, octyl methoxycinnamate, octyl salicylate, sulisobenzone, trolamine salicylate, avobenzone, terephthalylidene dicamphor sulfonic acid, titanium dioxide, zinc oxide, talc, 4-methylbenzylidene camphor, bisoctrizole, bis-ethylhexyloxyphenol methoxyphenol triazine, bisdisulizole disodium, drometrizole trisiloxane, sodium dihydroxy dimethoxy disulfobenzophenone, ethylhexyl triazone, diethylamino hydroxybenzoyl hexyl benzoate, diethylhexyl butamido triazone, dimethico-diethylbenzalmalonate, polysilicone-15, and isopentenyl-4-methoxycinnamate.

The personal care composition of the invention may also include one or more optical brighteners, such as, for example, a triazine-stilbenes (di-, tetra- or hexa-sulfonated), a coumarin, an imidazoline, a diazole, a triazole, a benzoxazoline, and a biphenyl stilbene.

In some embodiments, it may be preferred that the optical brightener(s) is a thiophene derivative, such as, for example, those having the following structure:

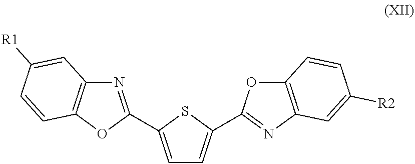

in which R¹ and R² are independently chosen from branched or unbranched, saturated or unsaturated alkyl radicals having 1 to 10 carbon atoms. A preferred thiophene derivative may include bis(t-butyl benzoxazolyl) thiophene, which is available from Inolex Chemical Company, Philadelphia, Pa., USA.

The invention includes personal care composition that are photostable, as compared to compositions containing identical ingredients, but which do not contain the polymer of the invention. For example, the photostable compositions of the invention are at least 50%, at least 40%, at least 30%, at least 20% and/or at least 10% more photostable compared to an identical formulation that does not contain the polymer of the invention. Present photostability comparison may be made using for example, the protocol set out in Example 3. Such photostable composition may include the polymer alone (where it acts to improve photostability of other compounds) or the polymer with one or more additional UV protective agent(s) (where it acts to improve the photostability of the additional agent(s) and other compounds).

Also included in the invention are synergistic compositions that contain the polymer of the invention and at least one additional UV protective agent. By “synergistic” it is meant that the SPF of the combined ingredients is greater than that expected when considering the SPF of the individual ingredients.

Also included within the scope of the invention are methods of protecting skin, hair, and/or nails of a mammal from damage caused by exposure to light in the UV wavelengths comprising applying to the skin, hair or nails a material the polymer described above and/or the personal care composition containing the polymer. “Skin” includes the external integument of living mammals, reptiles, amphibians, birds and other animals as well as processed skins, such as leathers or suedes. “Hair” includes hair, fur, wool and other filamentous keratinized structures of mammals and other animals. Similarly, “nails” includes claws, hooves and analogous structures of mammals and other animals.

Also within the scope of the invention are methods to improve the aesthetics of photoprotective formulations by allowing the exclusion of ingredients that may impart the feeling of oiliness and/or greasiness, or may impart a disagreeable odor.

Also included are methods photostabilizing a photoprotective personal care composition that contains a non-polymeric UV absorbing compound comprising incorporating into the composition an effective amount of the polymer(s) of the invention. In such methods, it may be preferred that the non-polymeric UV absorbing compound is selected from avobenzone, octylmethoxycinnamate and combinations thereof. Also included are methods of increasing the UV-A/UV-B ratio of a composition that contains a non-polymeric UV absorbing compound comprising incorporating into the composition an effective amount of the polymer of the invention. Methods of increasing the Sun Protection Factor of a photoprotective personal care composition that contains a non-polymeric UV absorbing compound are also included. Such methods include incorporating into the composition an effective amount of the polymer of the invention.

A method of increasing the UV-A protection provided by a photoprotective personal care composition that contains a non-polymeric UV absorbing compound comprising incorporating into the composition an effective amount of the polymer of the invention. In each of the methods, the comparative evaluation is carried out relative to a personal care composition that does not contain the polymer of the invention. Methods to evaluate the composition include the FDA Star Method, the COLIPA Guidelines, the Boots Method, and the Diffey Protocol.

### EXAMPLES

### Example 1

### Preparation of Inventive UV Absorbing Complex Polyester Polymer Containing a Benzophenone Group, a Naphthalene Group, and which can be Made Water Dispersible by Neutralization with a Base in Accordance with Scheme 1

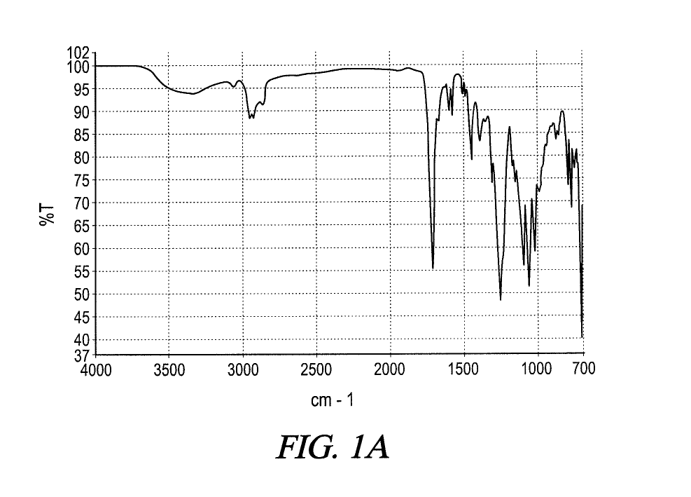

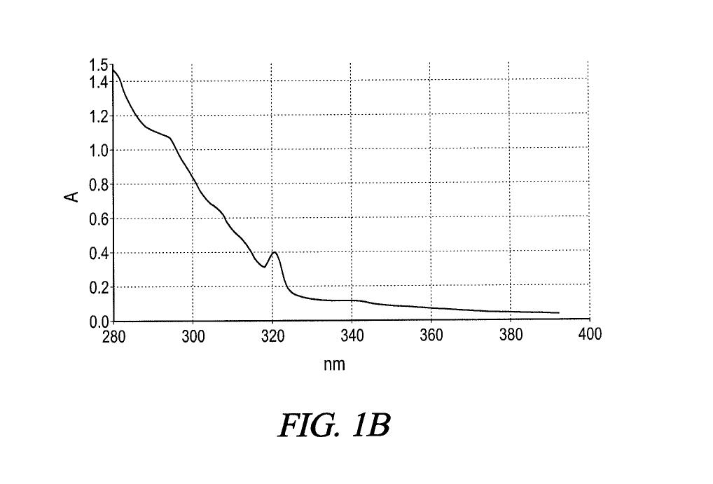

To a stirred batch round bottomed glass laboratory reactor with heating capability via an electrically heated mantle, inert gas sparging capability, vapor column, total condenser and receiver, 426 grams butylethylpropanediol (BEPD), and 840 grams of propylene glycol dibenzoate were added, the propylene glycol dibenzoate acting as a reaction solvent The mixture was heated to about 90° C., and 394 grams of benzophenone tetracarboxylic acid dianhydride (BTDA) were slowly added. The mixture was heated to about 135° C. and held until the acid value stalled indicating the completion of the anhydride ring-opening reaction between the BTDA and the BEPD leading to an acid functional UV absorbing complex polyester polymer containing a benzophenone group. No water of reaction evolved illustrating that the conditions were suitable for esterification by the opening of the anhydride rings in the BTDA only. To this polymer, 440 grams of naphthylglycidyl ether was added and the acid value was monitored until stall. The resulting polymer at 60% concentration in the solvent propylene glycol dibenzoate, Inventive UV Absorbing Complex Polyester Polymer B (UVACPPB) was cooled and discharged to a container. Table 1 shows the properties obtained. FIGS. 1A and 1B show the FTIR spectrum and the UV spectrum respectively. The properties of UVACPPB are shown in Table 1.

TABLE 1
Properties of UVACCPA2 and UVACCPA3

| Property | Value |
|---|---|
| Appearance | Yellow Viscous Liquid |
| Total Acid Number, mg KOH/g | 35.5 |
| Hydroxyl Number, mg KOH/g | 115.3 |
| Viscosity@80° C., cP | 1250 |

The polymer was dispersed in deionized water and heated to about 75° C. with agitation. Sodium hydroxide solution (2.0% wt/wt) was then slowly added until the pH reached about 7. The mixture was allowed to cool and what was resulted was stable milky dispersion. In this case, the acid groups in the polymer were converted to their respective sodium salts. Since the polymer contained the ester propylene glycol dibenzoate, it was demonstrated that the neutralized polymer acted as an effective emulsifier.

### Example 2

### Preparation of Inventive UV Absorbing Complex Polyester Polymers in Accordance with Schemes 7 and 6

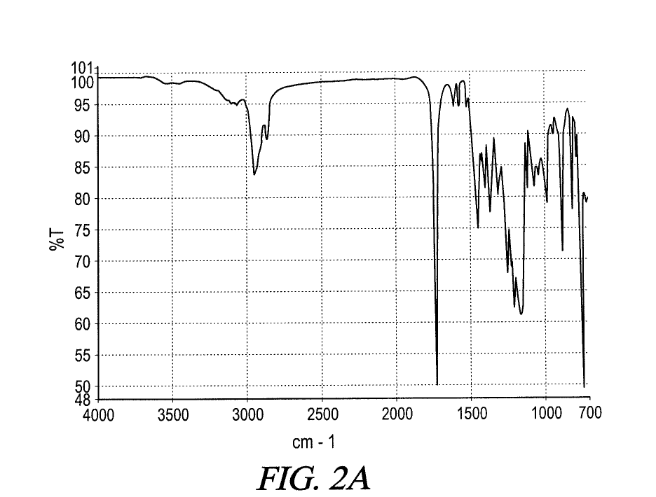

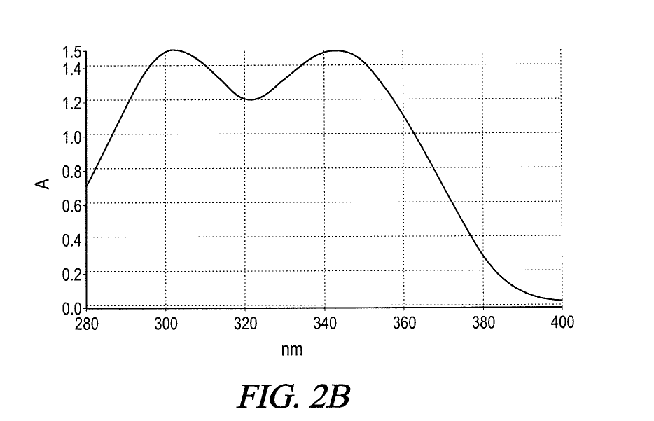

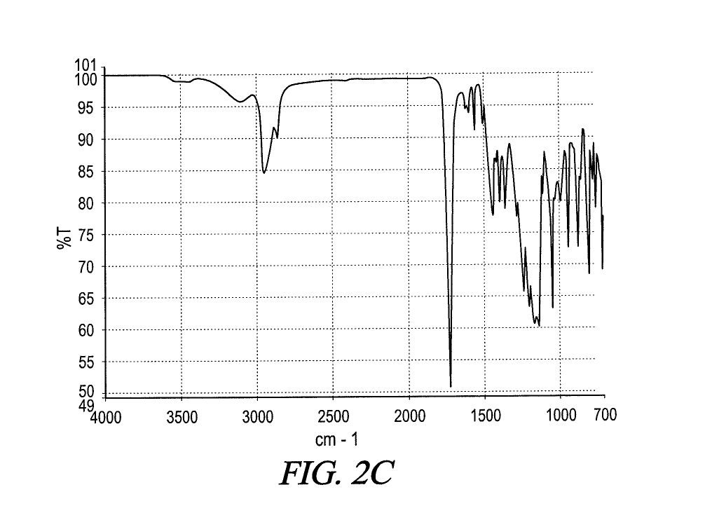

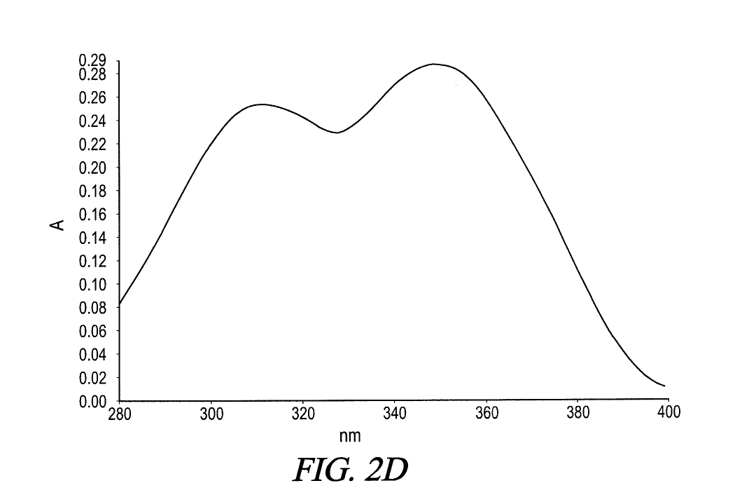

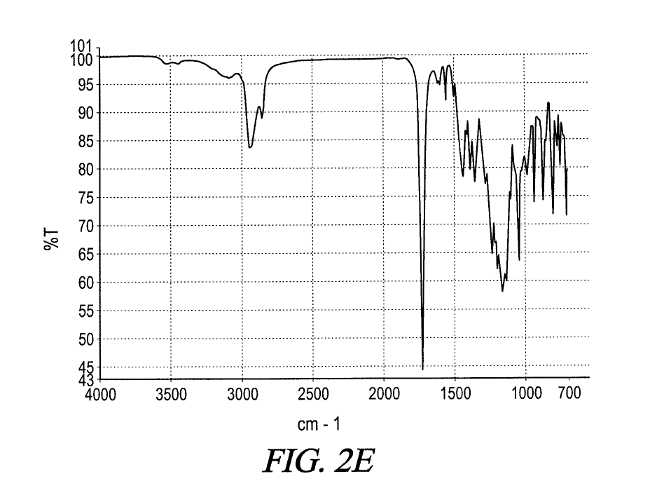

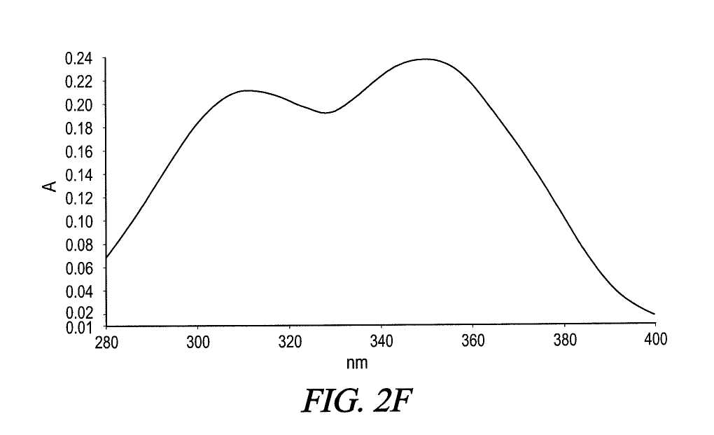

To prepare a linear UV absorbing complex polyester polymer in accordance with Scheme 7, to a stirred batch round bottomed glass laboratory reactor with heating capability via an electrically heated mantle, inert gas sparging capability, vapor column, total condenser and receiver, 584 grams of a mixture known as dibasic ester (“DBE”) consisting of methyl esters of hexanedioic acid, butanedioic acid, and pentanedioic acid in an approximate weight ratio of 1:1:3 were charged. To the reactor, 996 grams of 1,6-hexanediol were then charged. The mixture was heated to about 120° C., and 2,590 grams of benzenepropanoic acid, 3-(2H-benzotriazol-2-yl)-5-(1,1-dimethylethyl)-4-hydroxy-, methyl ester were than slowly added. A small quantity of transesterification catalyst was added, and the mixture was heated to about 230° C. As transesterification progressed, by-product methanol was collected in the receiver. When the theoretical quantity of methanol had been collected, the resulting polymer, Inventive UV Absorbing Complex Polyester Polymer A (UVACPPA) was cooled and discharged to a container. Table 2A shows the properties obtained. FIGS. 2A and 2B show the FTIR spectrum and the UV spectrum respectively.

TABLE 2A
Properties of UVACCPA.

| Property | UVACCPA Value |
|---|---|
| Appearance | Yellow Viscous Liquid |
| Color, Gardner | 11 |
| Total Acid Number, mg KOH/g | 0.54 |
| Hydroxyl Number, mg KOH/g | 32.2 |
| Viscosity@60° C., cP | 5,900 |
| Water Content, ppm | 100 |
| Molecular Weight, Daltons | 800 |

To prepare a cross linked UV absorbing complex polyester polymer in accordance with Scheme 6, to a round bottomed glass laboratory reactor with heating capability via an electrically heated mantle, inert gas sparging capability, vapor column, total condenser and receiver, 348 grams of dimethyl adipate, 236 grams of 1,6-hexanediol, 134 grams of trimethylolpropane were charged. The mixture was heated to about 100° C., then 775 grams of 3-(5-chloro-2H-benzotriazol-2-yl)-5-(1,1-dimethylethyl)-4-hydroxy-, methyl ester were charged. A small quantity of transesterification catalyst was added, and the mixture was heated to about 230° C. As transesterification progressed, by-product methanol was collected in the receiver. When the theoretical quantity of methanol had been collected, the resulting polymer, Inventive UV Absorbing Complex Polyester Polymer A2 (UVACPPA2) was cooled and discharged to a container. Table 1 shows the properties obtained. FIGS. 2C and 2D show the FTIR spectrum and the UV spectrum respectively.

To prepare a more highly crosslinked and higher molecular weight UV absorbing complex polyester polymer in accordance with Scheme 6, to the reaction set-up described above, 696 grams of dimethyl adipate, 472 grams of 1,6-hexanediol, 250 grams of di-trimethylolpropane were charged. The mixture was heated to about 100° C., then 1162.5 grams of 3-(5-chloro-2H-benzotriazol-2-yl)-5-(1,1-dimethylethyl)-4-hydroxy-, methyl ester were charged. A small quantity of transesterification catalyst was added, and the mixture was heated to about 220° C. As transesterification progressed, by-product methanol was collected in the receiver. When the theoretical quantity of methanol had been collected, the resulting polymer, Inventive UV Absorbing Complex Polyester Polymer A3 (UVACPPA3) was cooled and discharged to a container. Table 2B shows the properties obtained. FIGS. 2E and 2F show the FTIR spectrum and the UV spectrum respectively.

TABLE 2B
Properties of UVACCPA2 and UVACCPA3

| | UVACCPA2 | UVACCPA3 |
|---|---|---|
| Property | Value | Value |
| Appearance | Amber Viscous | Amber Viscous |
| Color, Gardner | 11 | 10 |
| Total Acid Number, mg KOH/g | 0.36 | 0.54 |
| Hydroxyl Number, mg KOH/g | 43 | 25 |
| Water Content, ppm | 87 | 103 |
| Molecular Weight, Daltons | 1300 | 2230 |

### Example 3

### Photostabilization Analysis Using Method of Stanfield

A test protocol has been developed and is widely used within the industry to test the photostability of sunscreens in-vitro (the method of Stanfield, et. al.) An index of photostability, β, has been developed and defined and is based on a model of the relationship between the applied UV dose and the UV dose transmitted by a typical sunscreen applied to a PMMA substrate. The sunscreen is irradiated and the UV absorbance is measured, before and at intervals during irradiation, and is used to compute the transmitted UV dose corresponding to each applied dose. The SPF is defined as the cumulative applied dose in MEDs (minimum erythemal dose,) when the transmitted dose reaches 1 MED (20 effective mJ/cm2). This corresponds to the SPF measured in the in-vivo test. Note that for a typical solar simulator a dose of 1 MED is approximately 2.45 J/cm2. A least-squares curve fit of applied UV dose vs. transmitted UV dose yields a power equation in the form:

y=αx^{β}

Since y=1 when x=SPF,

SPF=(1/α)^{1/β}

The initial SPF value is denoted by SPF₀, and represents the SPF value based on the initial absorbance of the sunscreen, theoretically before an UV dose has been administered. A completely photostable sunscreen would have a constant SPF equal to SPF₀. The value of β is set to 1/SPF₀. Then the value of β is determined as the value that satisfies the above equation with the known values of β (1/SPF₀) and SPF (from the SPF test on human subjects). The value of β is determined using the “Goal Seek” forecasting tool in Excel® (Microsoft, Redmond, Wash.). Based on a desirable value of at least 80% for SPF/SPF₀, the maximum acceptable values of β for a photostable sunscreen are shown in Table 3A.

TABLE 3A
Maximum Acceptable Values of β for an SPF/SPF₀ Ratio of At
Least 80 Percent

| SPF | Maximum Acceptable β |
|---|---|
| 8 | 1.10 |
| 15 | 1.09 |
| 30 | 1.07 |
| 40 | 1.06 |
| 50 | 1.06 |
| 80 | 1.05 |

Thus photostability may be characterized by the value of β for a given SPF or the ratio of SPF/SPF₀. Further detail concerning the theory and the test protocol may be found in the reference Stanfield J., Osterwalder U., Herzog B. “In vitro measurements of sunscreen protection. Photocem Photobiol Sci,” 2010, 9:489-494.

To test the photostabilization effect provided by inventive polymer UVACPPA, sunscreen formulations were prepared using the ingredient listed in Table 3B and the preparation procedure indicated below. All ingredient names conform to the nomenclature provided by the International Nomenclature of Cosmetics Ingredients (INCI) system, where applicable.

TABLE 3B
Control and test formulation utilizing UVPCCPA.

| | | Control | Sunscreen 3A, |
|---|---|---|---|
| | Ingredient (INCI Name) | % wt/wt. | % wt/wt. |
| Part A | Deionized Water | 61.15 | 55.15 |
| | Acrylates/C10-30 Alkyl | 0.30 | 0.30 |
| | Acrylate Crosspolymer | | |
| | Poloxamer 184 | 0.75 | 0.75 |
| Part B | Glycerin | 3.00 | 3.00 |
| | Disodium EDTA | 0.10 | 0.10 |
| Part C | Potassium Cetyl Phosphate | 3.00 | 3.00 |
| | Oxybenzone | 5.50 | 5.50 |
| | Avobenzone | 3.00 | 3.00 |
| | Neopentyl Glycol Diheptonate | 8.00 | 8.00 |
| | (and) Propylene Glycol | | |
| | Dibenzoate | | |
| | Octinoxate | 7.50 | 7.50 |
| | Octyl Salicylate | 5.00 | 5.00 |
| | UVPCCPA | — | 6.00 |
| | Stearic Acid | 1.50 | 1.50 |
| Part D | Aminomethyl Propanol | 0.20 | 0.20 |
| | Preservative | 1.00 | 1.00 |
| | Total | 100.00 | 100.00 |

All Parts below in refer to the components listed in Table 3B. The sunscreens were prepared by combining the components of Part A in a vessel and heating to 75° C. with propeller agitation until uniform. The ingredients of Part B were then added to Part A and propeller mixing continued. In a separate vessel, the components of Part C were combined and heated to 80° C. with propeller agitation until uniform. Part C was then added to the Part A/Part B blend and the mixture was homogenized at 3500 ppm for five minutes. The mixture than was allowed to cool to 45° C. with sweep mixing. The components of Part D were than added, and cooling and mixing continued until the temperature was 30° C. Mixing was ceased, and the sunscreen in the form of a cream was transferred to containers. In-vitro testing of sunscreens is conveniently performed using the Labsphere UV-2000S Transmittance Analyzer (Labsphere, North Sutton, N.H.) The function of the UV-2000S is to measure the transmittance and/or absorbance of ultraviolet (UV) radiation through sunscreen product and to compute internationally recognized effectiveness characteristics of the product. Operating instructions for the UV-2000S can be found in the operations manual “AQ-02755-000” dated Dec. 10, 2008 from Labsphere which is incorporated herein by reference. Within the operations manual, detailed instructions are provided related to the determination of transmittance, absorbance, and all previously defined numerical factors relating to in-vitro measurement of sun protection values utilizing the referenced testing protocols (COLIPA, Boots Star, and FDA method.)

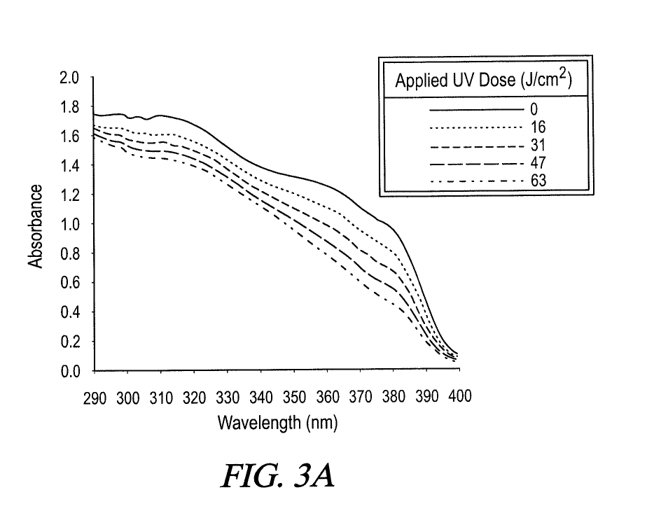

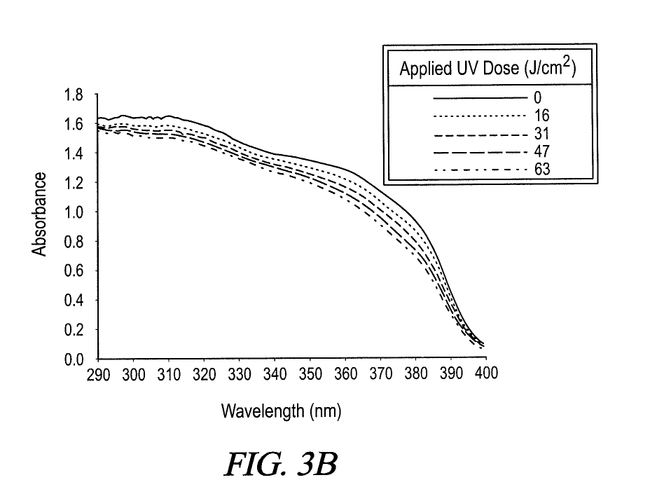

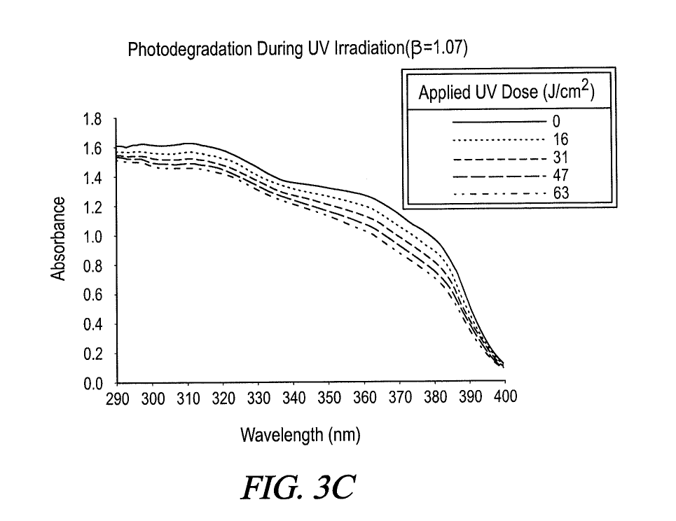

The static SPFᵢₙ₋ᵥᵢᵥₒ was determined on each of the formulations utilizing the previously referenced FDA method by Suncare Research Laboratories, LLC (Winston Salem, N.C., USA.). Photostability testing was then performed utilizing the method of Stanfield et. al. The sunscreen was applied at 0.75 mg/cm2 to 3 PMMA plates (Schönberg, Hamburg) and allowed to equilibrate for at least 15 minutes. A solar simulator, Model 16S (Solar Light Company, Philadelphia, Pa. USA) was used to irradiate the plates with a series of 5 UV doses, and the Labsphere UV-2000S Transmittance Analyzer was used to measure the sunscreen absorbance spectrum on each plate, before UV irradiation and after applied UV doses of 16, 31, 47 and 63 J/cm2 respectively. The measured absorbance values were adjusted by a factor β, with acceptable values between 0.8 and 1.2, so that the calculated SPF agreed with the in-vivo measured SPF. Then the transmitted UV dose vs. applied UV dose was graphed and the values of β, β and calculated SPF were determined, as described above. In addition, the absorbance spectrum corresponding to each UV dose was plotted to illustrate the degree of photodegradation at each wavelength during irradiation. These plots are provided in FIG. 3A for the Control and FIG. 3B for Sunscreen 3A. The numerical results are shown in Table 3C below:

TABLE 3C
Photostability results for Sunscreen 3A vs Control.

| | In-vivo | | Maximum | | | |
|---|---|---|---|---|---|---|
| | Measured | | Accept- | Measured | SPF/ | Photo- |
| Formula | Static SPF | χ | able β | β | SPF₀ | stable? |
| Control | 30.8 | 1.01 | 1.07 | 1.126 | 0.65 | No |
| Sunscreen | 32.1 | 0.89 | 1.07 | 1.06 | 0.81 | Yes |
| 3A | | | | | | |

The results indicate that a sunscreen containing the photo-unstable combination of AVO and OMC (Control) is very effectively photostabilized by the addition of inventive polymer UVACCBA. To further test the photostabilization effects that may be achieved through the use of the inventive polymers, an additional formulation was prepared. The ingredients are given in table 3D below.

TABLE 3D
Test formulation utilizing UVACCP3.

| | | Sunscreen 3B |
|---|---|---|
| | Ingredient (INCI Name) | % wt/wt. |
| Part A | Deionized Water | 55.15 |
| | Acrylates/C10-30 Alkyl | 0.30 |
| | Acrylate Crosspolymer | |
| | Poloxamer 184 | 0.75 |
| Part B | Glycerin | 3.00 |
| | Disodium EDTA | 0.10 |
| Part C | Potassium Cetyl Phosphate | 3.00 |
| | Oxybenzone | 5.50 |
| | Avobenzone | 3.00 |
| | Neopentyl Glycol Diheptonate | 8.00 |
| | (and) Propylene Glycol | |
| | Dibenzoate | |
| | Octinoxate | 7.50 |
| | Octisalate | 5.00 |
| | UVPCCPA3 | 6.00 |
| | Stearic Acid | 1.50 |
| Part D | Aminomethyl Propanol | 0.20 |
| | Preservative | 1.00 |
| | Total | 100.00 |

The formulations were prepared using the method described for the preparation of Sunscreen 3A, and were tested using the same method as that that was used for Sunscreen 3A. Again, In addition, the absorbance spectrum corresponding to each UV dose was plotted to illustrate the degree of photodegradation at each wavelength during irradiation. This plot is provided in FIG. 3C for Sunscreen 3B. Numerical results are provided in Table 3E.

TABLE 3E
Photostability results for Sunscreen 3B.

| | In vivo | | | | |
|---|---|---|---|---|---|
| | Measured | Maximum | | SPF/ | |
| Formula | Static SPF | Acceptable β | Measured β | SPF₀ | Photostable? |
| Sunscreen | 30 | 1.07 | 1.07 | 0.80 | Yes |
| 3B | | | | | |

### Example 4

### Determination of the SPF of the Inventive Substances in Absence of Other UV Filters

To determine the SPF provided by the inventive substances in the absence of other organic UV absorbers, the substances were formulated into a typical sunscreen emulsion in concentrations according to Table 4A.

TABLE 4A
Formulations for Testing SPF of Inventive Substances in the
Absence of Other UV Filters

| | | Sunscreen | Sunscreen | Sunscreen |
|---|---|---|---|---|
| | | 4A, | 4B, | 4C, |
| | Ingredient (INCI Name) | % wt/wt. | % wt/wt. | % wt/wt. |
| Part A | Deionized Water | 50.50 | 50.50 | 50.50 |
| | Acrylates/C10-30 Alkyl | 0.10 | 0.10 | 0.10 |
| | Acrylate Crosspolymer | | | |
| | Disodium EDTA | 0.10 | 0.10 | 0.10 |
| Part B | Propylene Glycol | 40.00 | 38.00 | 42.00 |
| | Dibenzoate | | | |
| | UVACPPA | 3.00 | — | — |
| | UVACPPB | — | 5.00 | — |
| | BBOT* | — | — | 1.00 |
| | Arachidyl Alcohol (and) | 4.00 | 4.00 | 4.00 |
| | Behenyl Alcohol (and) | | | |
| | Arachidyl Glucoside | | | |
| | Glyceryl Stearate (and) | 0.75 | 0.75 | 0.75 |
| | PEG-100 Stearate | | | |
| Part C | NaOH soln 10% | 0.75 | 0.75 | 0.75 |
| | Preservative | 0.80 | 0.80 | 0.80 |
| | Total | 100.00 | 100.00 | 100.00 |

*Bis(t-Butyl Benzoxazolyl) Thiophene

All Parts below refer to the components listed in Table 4A. The sunscreens were prepared by combining the components of Part A in a vessel and heating to 80° C. with propeller agitation. In a separate vessel, the components of Part B were combined and heated to 75° C. with propeller agitation. Part B was then added to Part A and the mixture was homogenized at 3500 ppm for five minutes. The mixture than was allowed to cool to 45° C. with sweep mixing. The components of Part C were than added, and cooling and mixing continued until the temperature was 30° C. Mixing was ceased, and the sunscreen in the form of a cream was transferred to containers. The SPFin-vitro, UVA/UVB Ratio and Critical Wavelength for the sunscreens were than determined utilizing the Labsphere UV-2000S using methods described previously.

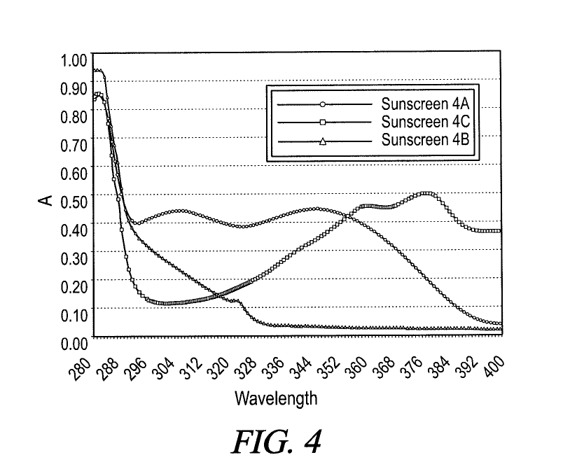

The results are shown in Table 4B. FIG. 4 shows the UV absorbance as a function of wavelength of each sample. The results show that while the inventive substances absorb UV radiation, they contribute very little to the SPFin-vitro in the absence of other UV filters at the tested use levels.

TABLE 4B
In-vitro test results for inventive substances in the absence
of other UV filters.

| Parameter | Sunscreen 4A | Sunscreen 4B | Sunscreen 4C |
|---|---|---|---|
| SPF_{in vitro} | 3 | 2 | 1 |
| UVA/UVB Ratio | 0.719 | 0.140 | 2.465 |
| Critical Wavelength | 371 | 356 | 391 |

### Example 5

### Surprising SPF Boosting Due to and Improvement in Aesthetics from the Inclusion of UVACPPA in Prototype Sunscreen Formulations

To evaluate the effectiveness of the inclusion of the inventive substance UVACPPA in actual prototype products, sunscreen formulations were prepared in accordance with the compositions shown in Table 5A. All ingredients other than the additional UV filters were the same and were used at similar levels as when the inventive substances were tested in the absence of additional UV filters (Example 4.)

TABLE 5A
Test formulation containing inventive UV absorbing complex
polyester polymer.
UVACPPA.

| | | Control | Sunscreen 5A, |
|---|---|---|---|
| | Ingredient (INCI Name) | % wt/wt. | % wt/wt. |
| Part A | Deionized Water | 50.5 | 50.5 |
| | Acrylates/C10-30 Alkyl | 0.1 | 0.1 |
| | Acrylate Crosspolymer | | |
| | Disodium EDTA | 0.1 | 0.1 |
| Part B | Homosalate | 15.0 | 15.0 |
| | Octisalate | 5.0 | 5.0 |
| | Octocrylene | 10.0 | 10.0 |
| | Oxybenzone | 5.0 | 5.0 |
| | Avobenzone | 3.0 | 3.0 |
| | NGDH | 2.0 | 2.0 |
| | Polyester-7 | 3.0 | 0.0 |
| | UVACPPA | 0.0 | 3.0 |
| | Arachidyl Alcohol (and) | 4.0 | 4.0 |
| | Behenyl Alcohol (and) | | |
| | Arachidyl Glucoside | | |
| | Glyceryl Stearate (and) | 0.75 | 0.75 |
| | PEG-100 Stearate | | |
| Part C | NaOH soln 10% | 0.75 | 0.75 |
| | Preservative | 0.8 | 0.8 |
| | Total | 100 | 100 |

All Parts below refer the ingredient shown in Table 5A. The sunscreens were prepared by combining the components of Part A in a vessel and heating to 80° C. with propeller agitation. In a separate vessel, the components of Part B were combined and heated to 75° C. with propeller agitation. Part B was then added to Part A and the mixture was homogenized at 3500 ppm for five minutes. The mixture than was allowed to cool to 45° C. with sweep mixing. The components of Part C were than added, and cooling and mixing continued until the temperature was 30° C. Mixing was ceased, and the sunscreen in the form of a cream was transferred to containers.

The SPF_{in vitro} and UVAPF were determined for the Control vs. Sunscreen 8A. Testing was conducted Suncare Research Laboratories, LLC (Winston-Salem N.C., USA) using the instrumentation and methods described previously. The data obtained is shown in Table 5B.

TABLE 5B
In-vitro test results for formulations of Table 5A.

| Parameter | Control | Sunscreen 5A |
|---|---|---|
| SPF_{in vitro} | 29.7 | 38.3 |
| UVAPF | 10.8 | 12.7 |

To determine the expected SPF that would be obtained from the inclusion of the inventive polymer UVACCPA, the Sunscreen Simulator was used. The Sunscreen Simulator is a computer model that enables the calculation of SPF, UVA/UVB-ratio, and critical wavelength. It is based on a step film model, by which inhomogeneities of the absorbing layer are introduced. The model reproduces synergistic effects on SPF induced by the presence of mixtures of UV-A and UV-B absorbing filters and can be used to design sunscreen formulations with a specific UV-A performance. The tool allows for the prediction of the SPF of a sunscreen based upon imputing concentrations of sunscreen filters. Further detail regarding the theory and methodology used in this in-silico modeling tool can be found in Herzog, B, Mendrok C, Mongiat S, Muller S, Osterwalder U, “The sunscreen simulator: A formulators tool to predict SPF and UVA parameters.” SOFW-Journal 2003:129:2-9. Although the results from the simulator are not a substitute for in-vitro and/or in-vivo sunscreen testing, it can provide some insight as to the expected changes in SPF that would result when various filter levels are increased, decreased, added, and/or removed. The simulator already includes extinction curves for globally approved sunscreens. Since extinction curves for the inventive polymers are not included in the simulator, curves for the polymers were compared to existing sunscreens, and the closest match was selected. The concentration of the existing sunscreen which resulted in an SPF of that provided by the inventive polymer (Table 4A) in the absence of other sun filters was then determined. Visual examination of the extinction curve for UVACPPA showed that the closest match was 2,2′-[6-(4-methoxyphenyl)-1,3,5-triazine-2,4-diyl]bis{5-[(2-ethylhexyl)oxy]phenol} (Bemotrizinol, Tinosorb S, BASF Corporation.) Using the simulator, the concentration of bemotrizinol required to provide an SPF of 3 in the absence of other filters (Table 4A) was determined and was found to be 0.95%. The types and levels of UV filters tested in the Control formulation of this example, 15.0% Homosalate, 5.0% Octisalate, 10.0% Octocrylene, 5.0% Oxybenzone, and 3.0% Avobenzone were then inputted into the simulator. The SPF calculated by the simulator was 37.0. A level of 0.95% Bemotrizinol was then inputted in addition to the aforementioned types and levels of filters. The resulting SPF was 40.5, an increase of 3.5 SPF units. Therefore, it was quite surprising that the inclusion of 3.0% inventive polymer boosted the SPF by about 9 SPF units when the simulator predicted an increase of only 3 based upon the Bemotrizinol model. This suggests that inventive polymer UVACPPA works synergistically with other non-polymeric chemical UV filters.

A third formulation was prepared in which both octisalate and homosalate were removed from the formulation. All other non-UV absorbing ingredients remained the same as in the preparations of the Control and Sunscreen 5A. The salicylate free formulation is shown in Table 5C.

TABLE 5C
Salicylate free formulation containing inventive UV absorbing complex
polyester polymer UVACPPA.

| | | Sunscreen 6A, |
|---|---|---|
| | Ingredient (INCI Name) | % wt/wt. |
| Part A | Deionized Water | 50.5 |
| | Acrylates/C10-30 Alkyl | 0.1 |
| | Acrylate Crosspolymer | |
| | Disodium EDTA | 0.1 |
| Part B | Homosalate | 0.0 |
| | Octisalate | 0.0 |
| | Octocrylene | 10.0 |
| | Oxybenzone | 5.0 |
| | Avobenzone | 3.0 |
| | NGDH | 22.0 |
| | Polyester-7 | 0.0 |
| | UVACPPA | 3.0 |
| | Arachidyl Alcohol (and) | 4.0 |
| | Behenyl Alcohol (and) | |
| | Arachidyl Glucoside | |
| | Glyceryl Stearate (and) | 0.75 |
| | PEG-100 Stearate | |
| Part C | NaOH soln 10% | 0.75 |
| | Preservative | 0.8 |
| | Total | 100 |

The formulation was prepared in the exact manner as described in the preparation of the Control and Sunscreen 5A of this Example. Table 5D shows in-vitro test results determined for Sunscreen 5B using the methodology described previously.

TABLE 5D
In-vitro test results for formulations of Table 5B.

| Parameter | Sunscreen 5B |
|---|---|
| SPF_{in vitro} | 29.7 |
| UVAPF | 12.1 |

The results show that by replacing 15.0% combined salicylates with 3.0% inventive polymer UVACPPA, the resulting in-vitro SPF obtained is the same as when the salicylates were present. Sunscreen 5A was evaluated for aesthetics vs. Control containing salicylates and was shown to be significantly less greasy, almost “dry” feeling, and odorless versus the pronounced salicylate odor of the Control.

Each of the Control and Sunscreen 5A were tested under FDA guidelines for static in-vivo SPF utilizing the FDA method described previously. For each of the Control and Sunscreen 5A, a five subject test panel was employed. Testing was conducted by Suncare Research Laboratories, LLC (Winston-Salem N.C., USA.) The results are provided in Table 5E.

TABLE 5E
In-vivo static SPF results for Control and Sunscreen 5A.

| Parameter | Control | Sunscreen 6A |
|---|---|---|
| SPFᵢₙ₋ᵥᵢᵥₒ (N = 5) | 31.2 | 41.2 |

The data shows that the inclusion of 3.0 percent UVACPPA resulted in an SPFᵢₙ₋ᵥᵢₜᵣₒ boost of 10 units over Control, thus validating in-vivo the surprising results obtained when the sunscreens were tested in-vitro.

### Example 6

### UV Evaluation of Inventive Polymer UVACPPB

To evaluate the potential effectiveness of the inclusion of UVACPPB in sunscreens, sunscreen oil phases were formulated in accordance with Table 6A.

TABLE 6A
Oil phase compositions for the UV evaluation of UVACPPB in
sunscreens.

| Ingredient (INCI Name) | Control, % wt./wt. | Blend 6A, % wt./wt. |
|---|---|---|
| Avobenzone | 0.17 | 0.17 |
| Octocrylene | 0.55 | 0.55 |
| Oxybenzone | 0.28 | 0.28 |
| UVACPPB | 0.00 | 0.33 |
| Nepentyl Glycol | 99.00 | 98.67 |
| Total | 100.00 | 100.00 |

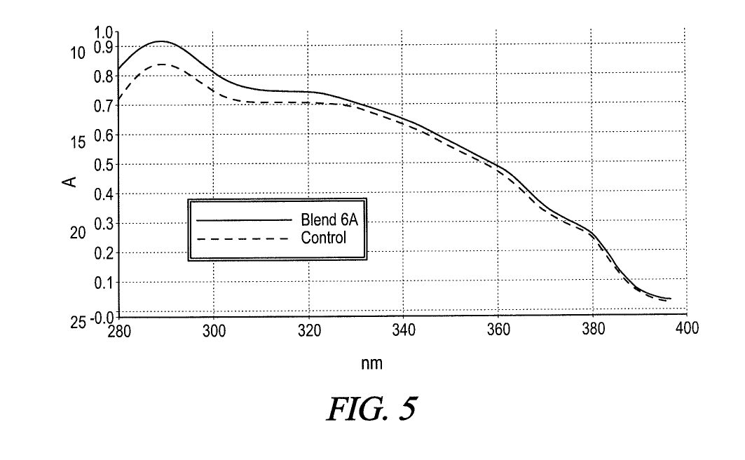

Each of the blends were diluted by weighing 200 mg of blend in a 100 mL volumetric flask and diluting to mark with tetrahydrofuran. The UV spectrum from 280 to 400 nm was then determined using a Perkin-Elmer Spectrum 100 UV/Visible Spectrophotometer. The results are found in FIG. 4. In FIG. 5, the curve with the stronger absorbance in the UV-B range is the result obtained from Blend 6A

### Example 7

### Surprising SPF Boosting Due to the Inclusion of UVACPPB in Prototype Sunscreen Formulations

To evaluate the effectiveness of the inclusion of the inventive substance UVACPPB in an actual prototype product, sunscreen formulations were prepared in accordance with the compositions shown in Table 7A.

TABLE 7A
Test formulations for the evaluation of the inclusion of UVACPPB in prototype
sunscreen formulations.

| | | Control 7A, | Sunscreen 7B, | Control 7C, | Sunscreen 7D, |
|---|---|---|---|---|---|
| | Ingredient (INCI Name) | % wt./wt. | % wt./wt. | % wt./wt. | % wt./wt. |
| A | Deionized Water | 52.55 | 52.55 | 52.55 | 52.55 |
| | Acrylates/C10-30 Alkyl | 0.10 | 0.10 | 0.10 | 0.10 |
| | Acrylate Crosspolymer | | | | |
| | Disodium EDTA | 0.10 | 0.10 | 0.10 | 0.10 |
| B | Octocrylene | 10.00 | 10.00 | 10.00 | 10.00 |
| | Oxybenzone | 6.00 | 6.00 | 0.00 | 0.00 |
| | Avobenzone | 3.00 | 3.00 | 3.00 | 3.00 |
| | Glyceryl Stearate (and) PEG- | 1.50 | 1.50 | 1.50 | 1.50 |
| | 100 Stearate | | | | |
| | Neopentyl Glycol Diheptanoate | 20.00 | 15.00 | 26.00 | 21.00 |
| | Arachidyl Alcohol (and) | 5.00 | 5.00 | 5.00 | 5.00 |
| | Behenyl Alcohol (and) | | | | |
| | Arachidyl Glucoside | | | | |
| | UVACPPB | 0.00 | 5.00 | 0.00 | 5.00 |
| C | Sodium Hydroxide, 10% | 0.75 | 0.75 | 0.75 | 0.75 |
| | Solution | | | | |
| | Preservative | 1.00 | 1.00 | 1.00 | 1.00 |
| | Total | 100.00 | 100.00 | 100.00 | 100.00 |

All Parts below refer the ingredient shown in Table 7A. The sunscreens were prepared by dispersing Acrylates/C10-30 Alkyl Acrylate Crosspolymer in a vortex of deionized water in a vessel. Then, the remaining components of Part A were added and heated to 80° C. with propeller agitation. In a separate vessel, the components of Part B were combined and heated to 75° C. with propeller agitation. Part B was then added to Part A and the mixture agitated until uniform. The mixture than was allowed to cool to 45° C. with sweep mixing. The components of Part C were than added, and cooling and sweep mixing continued until the temperature was 30° C. Mixing was ceased, and the sunscreen in the form of a cream was transferred to containers.

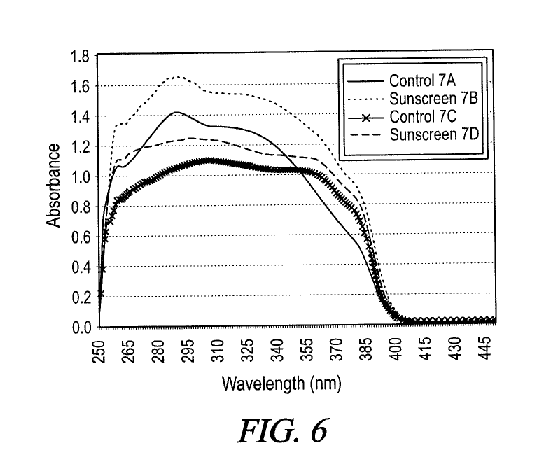

SPFᵢₙ₋ᵥᵢₜᵣₒ, UVA/UVB Ratio, and Critical Wavelength were determined on each of the Control formulations and Sunscreen formulations with the Labsphere UV-2000S using the methods described previously. FIG. 6 shows the absorbance as a function of wavelength for each of the four sunscreens. The data are summarized in Table 7B.

TABLE 7B
In-vitro data for control sunscreens vs. sunscreens containing
inventive UV absorbing complex polyester polymer UVACPPB.

| | Control | | | Sunscreen |
|---|---|---|---|---|
| Parameter | 7A | Sunscreen 7B | Control 7C | 7D |
| SPFᵢₙ₋ᵥᵢₜᵣₒ | 17.0 | 29.6 | 11.0 | 15.0 |
| UVA/UVB Ratio | 0.60 | 0.70 | 0.76 | 0.74 |
| Critical | 371 | 376 | 376 | 377 |
| Wavelength | | | | |

Surprisingly, although when tested in a sunscreen oil phase in the absence of other UV filters (Example 4,) inventive polymer UVACPPB contributed only 2 in-vitro SPF units (see Table 4A,) when formulated into an actual prototype formulations, the SPF was increased by 12.6 units and 4 units respectively. Furthermore, both the UVA/UVB ratio and the Critical Wavelength were increased despite the fact that UVACPPB absorbs mainly within the UV-B. This suggests that inventive polymer UVACPPB works synergistically with other UV filters, especially when oxybenzone is included in the formulation.

### Example 8

To evaluate the effectiveness of the inclusion of an inventive complex polyester polymers and/or an optical brightener to an actual prototype product, sunscreen formulations were prepared in accordance with the compositions shown in Table 8A.

TABLE 8A
Test formulations for the evaluation of the inclusion of an optical
brightener in a sunscreen.

| | | | | Sunscreen |
|---|---|---|---|---|
| | Ingredient (INCI | Control, | Sunscreen 8A, | 8B, |
| | Name) | % wt/wt. | % wt/wt. | % wt/wt. |
| Part A | Deionized Water | 50.50 | 50.50 | 50.50 |
| | Acrylates/C10-30 | 0.10 | 0.10 | 0.10 |
| | Alkyl Acrylate | | | |
| | Crosspolymer | | | |
| | Disodium EDTA | 0.10 | 0.10 | 0.10 |
| Part B | Homosalate | 15.00 | 15.00 | 15.00 |
| | Octisalate | 5.00 | 5.00 | 5.00 |
| | Octocrylene | 10.00 | 10.00 | 10.00 |
| | Oxybenzone | 5.00 | 5.00 | 5.00 |
| | Avobenzone | 3.00 | 3.00 | 3.00 |
| | NGDH | 2.00 | 0.00 | 4.00 |
| | Polyester-7 | 3.00 | 0.00 | 0.00 |
| | UVACPPA | 0.00 | 4.75 | 0.00 |
| | BBOT* | 0.00 | 0.25 | 1.00 |
| | Arachidyl Alcohol | 4.00 | 4.00 | 4.00 |
| | (and) Behenyl Alcohol | | | |
| | (and) Arachidyl | | | |
| | Glucoside | | | |
| | Glyceryl Stearate (and) | 0.75 | 0.75 | 0.75 |
| | PEG-100 Stearate | | | |
| Part C | NaOH soln 10% | 0.75 | 0.75 | 0.75 |
| | Preservative | 0.80 | 0.80 | 0.80 |
| | Total | 100.00 | 100.00 | 100.00 |

*Bis(t-Butyl Benzoxazolyl) Thiophene

All Parts below refer the ingredient shown in Table 8A. The sunscreens were prepared by combining the components of Part A in a vessel and heating to 80° C. with propeller agitation. In a separate vessel, the components of Part B were combined and heated to 75° C. with propeller agitation. Part B was then added to Part A and the mixture was homogenized at 3500 ppm for five minutes. The mixture than was allowed to cool to 45° C. with sweep mixing. The components of Part C were than added, and cooling and mixing continued until the temperature was 30° C. Mixing was ceased, and the sunscreen in the form of a cream was transferred to containers.

SPFᵢₙ₋ᵥᵢₜᵣₒ, UVA/UVB Ratio, and Critical Wavelength were determined for each of the Control formulation and Sunscreen formulations with the Labsphere UV-2000S using the methods described previously. FIG. 7 shows the absorbance as a function of wavelength for each of the three sunscreens. The data are summarized in Table 8B.

TABLE 8B
In-vitro data for control sunscreens vs. sunscreens containing
inventive UV absorbing complex polyester polymer UVACPPB.

| | | Sunscreen | Sunscreen |
|---|---|---|---|
| Parameter | Control | 8A | 8B |
| SPF_{in vitro} | 25.3 | 38.7 | 39.2 |
| UVA/UVB Ratio | 0.64 | 0.75 | 0.76 |
| Critical Wavelength | 372.4 | 376.4 | 380.8 |

The results show that the inclusion of 4.75% UVACPPA and 0.25% BBOT increased the SPF by 13.4 units, significantly more than that predicted from the results provided in Example 4. Furthermore, the results show that the inclusion of 1.0% BBOT in the absence of the polymer increases the SPF by 13.9 units while it was shown in Example 4 that use of 1.0% BBOT alone contributes 1 SPF unit.

As can been seen from the data provided in Table 8B, the combination of the optical brightener and the polymer of the invention provides a composition that exhibits an increased UV-A/UV-B ratio and an increased critical wavelength as compared to the composition containing the optical brightener alone.

### Example 9

To prepare a more highly crosslinked and higher molecular weight UV absorbing complex polyester polymer in accordance with Scheme 6 that has a limited number of low molecular weight oligomers, to a stirred batch round bottomed glass laboratory reactor with heating capability via an electrically heated mantle, inert gas sparging capability, vapor column, total condenser and receiver, 696 grams of dimethyl adipate, 1301 grams of dimerdiol, and 775 grams of di-trimethylolpropane were charged. The mixture was heated to about 100° C., then 2824 grams of benzenepropanoic acid, 3-(2H-benzotriazol-2-yl)-5-(1,1-dimethylethyl)-4-hydroxy-, methyl were charged. A small quantity of transesterification catalyst was added, and the mixture was heated to about 200° C. As transesterification progressed, by-product methanol was collected in the receiver. When the theoretical quantity of methanol had been collected, the resulting polymer, Inventive UV Absorbing Complex Polyester Polymer A4 (UVACPPA4) was cooled and discharged to a container. GPC analysis was performed using right angle light scattering detection. Table 9 shows the properties obtained.

TABLE 9
Properties of UVACCPA4.

| Property | UVACCPA4 Value |
|---|---|
| Appearance | Amber Viscous |
| Color, Gardner | 13 |
| Total Acid Number, mg KOH/g | 0.15 |
| Hydroxyl Number, mg KOH/g | 13.6 |
| Water Content, ppm | 160 |
| Molecular Weight (Mn), Daltons, by GPC | 2,670 |
| Molecular Weight (Mw) Daltons), by GPC | 5,180 |
| Molecular Weight (Mz) Daltons), by GPC | 14,590 |
| Polydispersity Index | 1.94 |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. An UV absorbing complex polyol polyester polymer that is the product of a reaction scheme comprising:
(i) the esterification of a polyol and a dianhydride, wherein the esterification is carried out under conditions that facilitate substantially only anhydride opening, to form a polyester polymer comprising at least two pendant carboxylic groups, and at least two hydroxyl groups; and
(ii) the reaction of at least one pendant carboxylic group and at least one terminal hydroxyl group of the polyester polymer with an epoxide having a functional group, wherein the epoxide comprises an UV absorbing moiety.

2. The UV absorbing complex polyol polyester polymer according to claim 1, wherein:
the polyol is a diol, and
the dianhydride is UV absorbing and comprises a benzophenone moiety,
wherein the esterification step of (i) yields a polyester polymer comprising a pendent carboxylic acid and a terminal hydroxyl group as represented by Formula (IX): 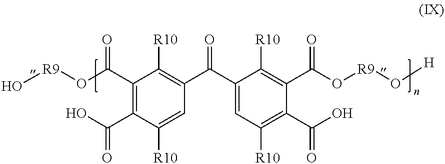
wherein R⁹ is independently selected from a hydrocarbon group having 2 to 54 carbon atoms, and 0 to 30 ether linkages, R¹⁰ is independently —H, or —OH, and n is an integer of 1 to 1000.

3. The polymer of claim 2, wherein the reaction of step (ii) comprises the etherification reaction of the functional group of the expoxide with the hydroxyl and/or carboxylic acid groups of the polyester polymer.

4. The polymer of claim 1, wherein:
the polyol is a diol, and
the dianhydride is not UV absorbing,
wherein the esterification step of (i) yields a polyester polymer comprising at least two pendant carboxylic acid groups and two terminal hydroxyl groups represented by Formula (X) 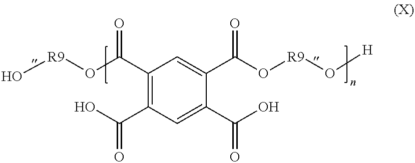
wherein R⁹ is independently selected from a hydrocarbon group having 2 to 54 carbon atoms, and 0 to 30 ether linkages, and n is an integer of 1 to 1000.

5. The polymer of claim 4, wherein the reaction of step (ii) comprises the etherification reaction of the functional group of the expoxide with at least one of the hydroxyl and/or carboxylic acid groups of the polyester polymer.

6. The polymer of claim 1, wherein the epoxide is derived from the epoxidation of an UV absorbing alcohol.

7. The polymer of claim 1, wherein the epoxide is derived from the epoxidation of an UV absorbing carboxylic acid.

8. The polymer of claim 1, wherein the epoxide is derived from a reaction represented by the reaction scheme 8A: 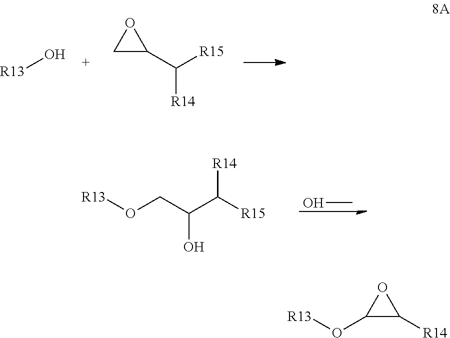
wherein R¹³ comprises an UV absorbing moiety, and R¹⁴ is independently selected from a hydrogen atom, and a hydrocarbon group having 1 to 54 carbon atoms and 0 to 30 ether linkages, and R¹⁵ is an halogen atom.

9. The polymer of claim 1, wherein the epoxide is derived from an UV absorbing carboxylic acid and from a reaction represented by the reaction scheme: 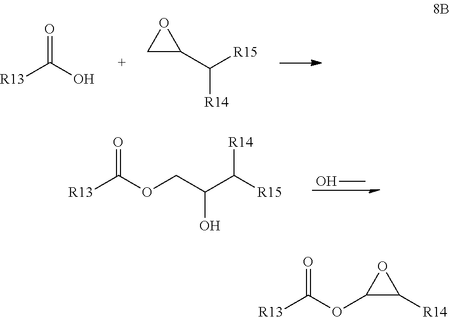
wherein R¹³ comprises an UV absorbing moiety, and R¹⁴ is independently selected from a hydrogen atom, and a hydrocarbon group having 1 to 54 carbon atoms and 0 to 30 ether linkages, and R¹⁵ is an halogen atom.

10. The polymer of claim 1, wherein the UV absorbing moiety of the epoxide is selected from a derivatized benzophenone moiety, derivatized naphthalene moiety, and a benzotriazole derivative.

11. The polymer of claim 1, wherein the UV absorbing moiety of the epoxide is selected from bis-ethylhexyloxyphenol methoxyphenyl triazine; butyl methoxydibenzoylmethane; diethylamino hydroxybenzoyl hexyl benzoate; disodium phenyl dibenzimidazole tetrasulfonate; drometrizole trisiloxane; methylene bis-benzotriazolyl tetramethylbutylphenol; and derivatives thereof.

12. The polymer of claim 1, wherein the UV absorbing moiety of the epoxide is selected from terephthalylidene dicamphor sulfonic acid; menthyl anthranilate; methylene bis-benzotriazolyl tetramethylbutylphenol; 4-methylbenzylidene camphor; benzophenone-3; benzophenone-4; diethylhexyl butamido triazone; ethylhexyl methoxycinnamate; and derivatives thereof.

13. The polymer of claim 1, wherein the UV absorbing moiety of the epoxide is selected from ethylhexyl salicylate; ethylhexyl triazone; ethylhexyl dimethyl PABA; homomethyl salicylate; isoamyl p-methoxycinnamate; octocrylene; phenylbenzimidazol sulfonic acid; polysilicone-15; benzotriazolyl dodecyl p-cresol; butyloctyl salicylate; diethylhexyl 2,6-naphthalate; diethylhexyl syringylidene malonate and polyester-8, and derivatives thereof.

14. A personal care composition comprising an UV absorbing complex polyol polyester polymer that is the product of a reaction scheme comprising: (i) the esterification of a polyol and a dianhydride, wherein the esterification is carried out under conditions that facilitate substantially only anhydride opening, to form a polyester polymer comprising at least two pendant carboxylic groups, and at least two hydroxyl groups; and (ii) the reaction of at least one pendant carboxylic group and at least one terminal hydroxyl group of the polyester polymer with an epoxide having a functional group, wherein the epoxide comprises an UV absorbing moiety.

15. The composition of claim 14, further comprising one or more components selected from a vegetable oil, a surfactant, a lipid, an alcohol, a wax, a pigments, a vitamin, a fragrance, a bleaching agent, an antibacterial agent, an anti-inflammatory agent, an antimycotic agent, a thickener, a gum, a starch, a chitosan, a polymeric material, a cellulosic material, a glycerin, a protein, an amino acid, a keratin fiber, a fatty acid, a siloxane, a botanical extract, an abrasive, a chemical exfoliant, a mechanical exfoliant, an anticaking agent, an antioxidant agent, a binder, a clay, a biological additive, a buffering agent, a bulking agent, a chelating agent, a film former, an humectant, an opacifying agent, a pH adjuster, a preservative, a propellant, a reducing agent, a skin darkening agent, an essential oils, a skin sensates, and combinations of these.

16. The composition of claim 14, further comprising an further comprising an optical brightener.

17. The composition of claim 16, wherein the optical brightener is selected from a triazine-stilbenes (di-, tetra- or hexa-sulfonated), a coumarin, an imidazoline, a diazole, a triazole, a benzoxazoline, and a biphenyl stilbene.

18. The composition of claim 16, wherein the optical brightener is a thiophene derivative.

19. The composition of claim 16, wherein the optical brightener is bis(t-butyl benzoxazolyl) thiophene.

20. The composition of claim 16, wherein the optical brightener is represented by the formula (XII) 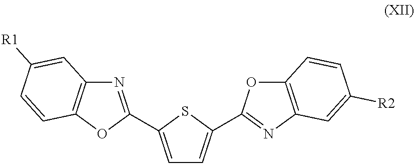
in which R¹ and R² are independently chosen from branched or unbranched, saturated or unsaturated alkyl radicals having 1 to 10 carbon atoms.

21. The composition of claim 14, wherein:
the polyol is a diol, and
the dianhydride is UV absorbing and comprises a benzophenone moiety,
wherein the esterification step of (i) yields a polyester polymer comprising a pendent carboxylic acid and a terminal hydroxyl group as represented by Formula (IX): 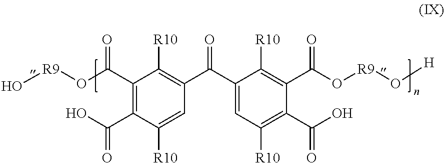
wherein R⁹ is independently selected from a hydrocarbon group having 2 to 54 carbon atoms, and 0 to 30 ether linkages, R¹⁰ is independently —H, or —OH, and n is an integer of 1 to 1000.

22. The composition of claim 14, wherein the reaction of step (ii) comprises the etherification reaction of the functional group of the expoxide with the hydroxyl and/or carboxylic acid groups of the polyester polymer.

23. The composition of claim 14, the polyol is a diol, and the dianhydride is not UV absorbing, wherein the esterification step of (i) yields a polyester polymer comprising at least two pendant carboxylic acid groups and two terminal hydroxyl groups represented by Formula (X): 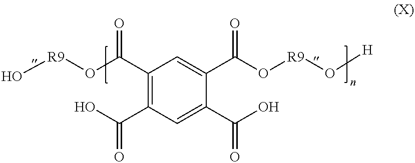
wherein R⁹ is independently selected from a hydrocarbon group having 2 to 54 carbon atoms, and 0 to 30 ether linkages, and n is an integer of 1 to 1000.

24. The composition of claim 14, wherein the reaction of step (ii) comprises the etherification reaction of the functional group of the expoxide with at least one of the hydroxyl and/or carboxylic acid groups of the polyester polymer.

25. The composition of claim 14, wherein the epoxide is derived from the epoxidation of an UV absorbing alcohol.

26. The composition of claim 14, wherein the epoxide is derived from the epoxidation of an UV absorbing carboxylic acid.

27. The composition of claim 14 further comprising at least one non non-polymeric UV absorbing compound.

28. The method of claim 27, wherein the non-polymeric UV absorbing compound is selected from avobenzone, octylmethoxycinnamate and combinations thereof.

29. A method of increasing the photostability of a personal care composition comprising incorporating the UV absorbing complex polyol polyester polymer of claim 1 into a personal care composition.

30. A method of protecting a portion of the skin, hair and or nails of a mammal from damage by UV light comprising applying an effective amount of the UV absorbing complex polyol polyester polymer of claim 1 to the portion of skin, hair or nails.

31. A method of photostabilizing a photoprotective personal care composition that contains a non-polymeric UV absorbing compound comprising incorporating into the composition an effective amount of the polymer of claim 1.

32. The method of claim 31, wherein the non-polymeric UV absorbing compound is selected from avobenzone, octylmethoxycinnamate and combinations thereof.

33. A method of increasing the UV-A/UV-B ratio of a composition that contains a non-polymeric UV absorbing compound comprising incorporating into the composition an effective amount of the polymer of claim 1, wherein the increase in protection is evaluated using the Boots Method.

34. A method of increasing the Sun Protection Factor of a photoprotective personal care composition that contains a non-polymeric UV absorbing compound comprising incorporating into the composition an effective amount of the polymer of claim 1.

35. The method of claim 34, wherein the non-polymeric UV absorbing compound is selected from avobenzone, octylmethoxycinnamate and combinations thereof.

36. A method of increasing the UV-A protection provided by a photoprotective personal care composition that contains a non-polymeric UV absorbing compound comprising incorporating into the composition an effective amount of the polymer of claim 1.

37. The method of claim 36, wherein the non-polymeric UV absorbing compound is selected from avobenzone, octylmethoxycinnamate and combinations thereof.

38. The method of claim 36, wherein the UV-A protection provided is evaluated using a method chosen from the FDA Star Method, the COLIPA Guidelines, the Boots Method, and the Diffey Protocol.

39. A linear UV absorbing complex polyol polyester polymer represented by Formula (XI): 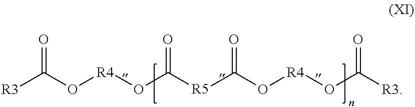
wherein R³ is independently selected from an UV absorbing moiety; R⁴ and R⁵ are each independently selected from a hydrocarbon group, and n is an integer of 1 to 1000.

40. The polymer of claim 39, wherein the UV absorbing moiety is chosen from a compound containing an UV absorbing benzotriazole group.

41. The polymer of claim 39, wherein the UV absorbing benzotriazole group is represented by the structure (Ia): 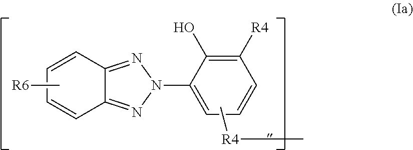
wherein R⁶ is independently an hydrogen atom or an halogen atom, and R⁴ is a hydrocarbon group.

42. The polymer of claim 39, wherein the UV-absorbing benzotriazole group is chosen from benzenepropanoic acid, 3-(2H-benzotriazol-2-yl)-5-(1,1-dimethylethyl)-4-hydroxy-, alkyl ester; benzenepropanoic acid, 3-(2H-benzotriazol-2-yl)-5-(1,1-dimethylethyl)-4-hydroxy-; benzenepropanoic acid, 3-(5-chloro-2H-benzotriazol-2-yl)-5-(1,1-dimethylethyl)-4-hydroxy-, alkyl ester and; 3-(5-chloro-2H-benzotriazol-2-yl)-5-(1,1-dimethylethyl)-4-hydroxy-, and/or derivatives thereof.

43. The polymer of claim 39, wherein the benzenepropanoic acid, 3-(2H-benzotriazol-2-yl)-5-(1,1-dimethylethyl)-4-hydroxy-, alkyl ester and/or benzenepropanoic acid, 3-(5-chloro-2H-benzotriazol-2-yl)-5-(1,1-dimethylethyl)-4-hydroxy-, alkyl ester are each independently methyl esters.

44. The polymer of claim 39, wherein R⁴ and R⁵ are each independently selected from a hydrocarbon group having 2 to 54 carbon atoms, and 0 to 30 ether linkages, wherein each of the carbons of the hydrocarbon group is independently substituted or unsubstituted, and saturated or unsaturated.

45. A personal care composition comprising a linear UV absorbing complex polyol polyester polymer represented by Formula (XI): 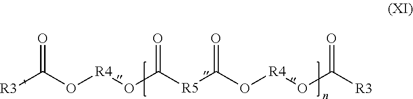
wherein R³ is independently selected from an UV absorbing moiety; R⁴ and R⁵ are each independently selected from a hydrocarbon group, and n is an integer of 1 to 1000.

46. The composition of claim 45, wherein the UV absorbing moiety is chosen from a compound containing an UV absorbing benzotriazole group.

47. The composition of claim 46, wherein the UV absorbing benzotriazole group is represented by the structure (Ia): 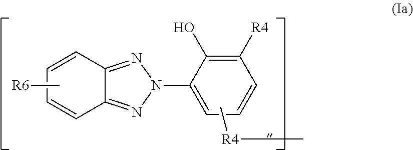
wherein R⁶ is independently an hydrogen atom or an halogen atom, and R⁴ is a hydrocarbon group.

48. The composition of claim 46, wherein the UV-absorbing benzotriazole group is chosen from benzenepropanoic acid, 3-(2H-benzotriazol-2-yl)-5-(1,1-dimethylethyl)-4-hydroxy-, alkyl ester; benzenepropanoic acid, 3-(2H-benzotriazol-2-yl)-5-(1,1-dimethylethyl)-4-hydroxy-; benzenepropanoic acid, 3-(5-chloro-2H-benzotriazol-2-yl)-5-(1,1-dimethylethyl)-4-hydroxy-, alkyl ester and; 3-(5-chloro-2H-benzotriazol-2-yl)-5-(1,1-dimethylethyl)-4-hydroxy-, and/or derivatives thereof.

49. The composition of claim 46, wherein the benzenepropanoic acid, 3-(2H-benzotriazol-2-yl)-5-(1,1-dimethylethyl)-4-hydroxy-, alkyl ester and/or benzenepropanoic acid, 3-(5-chloro-2H-benzotriazol-2-yl)-5-(1,1-dimethylethyl)-4-hydroxy-, alkyl ester are each independently methyl esters.

50. The composition of claim 45, wherein R⁴ and R⁵ are each independently selected from a hydrocarbon group having 2 to 54 carbon atoms, and 0 to 30 ether linkages, wherein each of the carbons of the hydrocarbon group is independently substituted or unsubstituted, and saturated or unsaturated.

51. The composition of claim 45 further comprising one or more components selected from a vegetable oil, a surfactant, a lipid, an alcohol, a wax, a pigments, a vitamin, a fragrance, a bleaching agent, an antibacterial agent, an anti-inflammatory agent, an antimycotic agent, a thickener, a gum, a starch, a chitosan, a polymeric material, a cellulosic material, a glycerin, a protein, an amino acid, a keratin fiber, a fatty acid, a siloxane, a botanical extract, an abrasive, a chemical exfoliant, a mechanical exfoliant, an anticaking agent, an antioxidant agent, a binder, a clay, a biological additive, a buffering agent, a bulking agent, a chelating agent, a film former, an humectant, an opacifying agent, a pH adjuster, a preservative, a propellant, a reducing agent, a skin darkening agent, an essential oils, a skin sensates, and combinations of these.

52. The composition of claim 45, further comprising an optical brightener.

53. The composition of claim 45, wherein the optical brightener is chosen from a triazine-stilbenes (di-, tetra- or hexa-sulfonated), a coumarin, an imidazoline, a diazole, a triazole, a benzoxazoline, and a biphenyl stilbene.

54. The composition of claim 45, wherein the optical brightener is a thiophene derivative.

55. The composition of claim 45, wherein the optical brightener is bis(t-butyl benzoxazolyl) thiophene.

56. The composition of claim 45, wherein the optical brightener is represented by the formula: 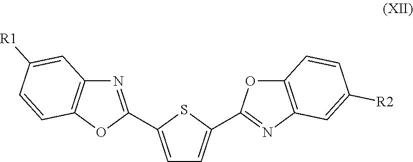
in which R¹ and R² are independently chosen from branched or unbranched, saturated or unsaturated alkyl radicals having 1 to 10 carbon atoms.

57. A method of increasing the photostability of a personal care composition comprising incorporating the linear UV absorbing complex polyol polyester polymer of claim 34 into a personal care composition.

58. A method of protecting a portion of the skin, hair and or nails of a mammal from damage by UV light comprising applying an effective amount of the linear UV absorbing complex polyol polyester polymer of claim 39 to the portion of skin, hair or nails.

59. A method of photostabilizing a photoprotective personal care composition that contains a non-polymeric UV absorbing compound comprising incorporating into the composition an effective amount of the linear UV absorbing complex polyol polyester polymer of claim 39.

60. The method of claim 59, wherein the non-polymeric UV absorbing compound is selected from avobenzone, octylmethoxycinnamate and combinations thereof.

61. A method of increasing the UV-A/UV-B ratio of a composition that contains a non-polymeric UV absorbing compound comprising incorporating into the composition an effective amount the linear UV absorbing complex polyol polyester polymer of claim 39, wherein the increase in protection is evaluated using the Boots Method.

62. A method of increasing the Sun Protection Factor of a photoprotective personal care composition that contains a non-polymeric UV absorbing compound comprising incorporating into the linear UV absorbing complex polyol polyester polymer of claim 39.

63. The method of claim 62, wherein the non-polymeric UV absorbing compound is selected from avobenzone, octylmethoxycinnamate and combinations thereof.

64. A method of increasing the UV-A protection provided by a photoprotective personal care composition that contains a non-polymeric UV absorbing compound comprising incorporating into the composition an effective amount of the linear UV absorbing complex polyol polyester polymer of claim 39.

65. The method of claim 64, wherein the non-polymeric UV absorbing compound is selected from avobenzone, octylmethoxycinnamate and combinations thereof.

66. The method of claim 65, wherein the UV-A protection provided is evaluated using a method chosen from the FDA Star Method, the COLIPA Guidelines, the Boots Method, and the Diffey Protocol.

67. A crosslinked UV absorbing complex polyol polyester polymer that is reaction product of a random copolyesterification esterification reaction and/or the esterification product of:
a monofunctional carboxylic acid and/or ester that comprises an UV absorbing moiety,
at least one of a diol, a polyol, a diacid and/or an ester,
wherein the polymer has an UV absorbing functionality of greater than 2.0.

68. The polymer of claim 67 wherein the monofunctional carboxylic acid and/or ester is represented by Formula (I): 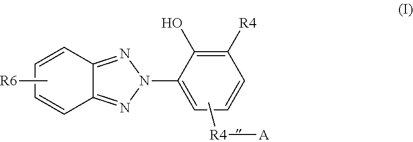
wherein R⁶ is independently selected from a hydrogen atom or a halogen atom, R⁴ is a hydrocarbon group, and A is a functional group selected from the group consisting of carboxylic acid and ester.

69. A personal care composition comprising a crosslinked UV absorbing complex polyol polyester polymer that is reaction product of a random copolyesterification esterification reaction and/or the esterification product of:
a monofunctional carboxylic acid and/or ester that comprises an UV absorbing moiety,
at least one of a diol, a polyol, a diacid and/or an ester,
wherein the polymer has an UV absorbing functionality of greater than 2.0.

70. The composition of claim 68, further comprising one or more components selected from a vegetable oil, a surfactant, a lipid, an alcohol, a wax, a pigments, a vitamin, a fragrance, a bleaching agent, an antibacterial agent, an anti-inflammatory agent, an antimycotic agent, a thickener, a gum, a starch, a chitosan, a polymeric material, a cellulosic material, a glycerin, a protein, an amino acid, a keratin fiber, a fatty acid, a siloxane, a botanical extract, an abrasive, a chemical exfoliant, a mechanical exfoliant, an anticaking agent, an antioxidant agent, a binder, a clay, a biological additive, a buffering agent, a bulking agent, a chelating agent, a film former, an humectant, an opacifying agent, a pH adjuster, a preservative, a propellant, a reducing agent, a skin darkening agent, an essential oils, a skin sensates, and combinations of these.

71. The composition of claim 69, further comprising an further comprising an optical brightener.

72. The composition of claim 71, wherein the optical brightener is selected from a triazine-stilbene (di-, tetra- or hexa-sulfonated), a coumarin, an imidazoline, a diazole, a triazole, a benzoxazoline, and a biphenyl stilbene.

73. The composition of claim 71, wherein the optical brightener is bis(t-butyl benzoxazolyl) thiophene.

74. The composition of claim 71, wherein the optical brightener is represented by the formula (XII): 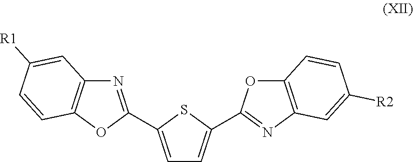
in which R¹ and R² are independently chosen from branched or unbranched, saturated or unsaturated alkyl radicals having 1 to 10 carbon atoms.

75. A method of increasing the photostability of a personal care composition comprising incorporating the UV absorbing complex polyol polyester polymer of claim 69 into a personal care composition.

76. A method of protecting a portion of the skin, hair and or nails of a mammal from damage by UV light comprising applying an effective amount of the UV absorbing complex polyol polyester polymer of claim 69 to the portion of skin, hair or nails.

77. A method of photostabilizing a photoprotective personal care composition that contains a non-polymeric UV absorbing compound comprising incorporating into the composition an effective amount of the polymer of claim 69.

78. The method of claim 77, wherein the non-polymeric UV absorbing compound is selected from avobenzone, octylmethoxycinnamate and combinations thereof.

79. A method of increasing the UV-A/UV-B ratio of a composition that contains a non-polymeric UV absorbing compound comprising incorporating into the composition an effective amount of the polymer of claim 69, wherein the increase in protection is evaluated using the Boots Method.

80. A method of increasing the Sun Protection Factor of a photoprotective personal care composition that contains a non-polymeric UV absorbing compound comprising incorporating into the composition an effective amount of the polymer of claim 69.

81. The method of claim 80 wherein the non-polymeric UV absorbing compound is selected from avobenzone, octylmethoxycinnamate and combinations thereof.

82. A method of increasing the UV-A protection provided by a photoprotective personal care composition that contains a non-polymeric UV absorbing compound comprising incorporating into the composition an effective amount of the polymer of claim 69.

83. The method of claim 82, wherein the non-polymeric UV absorbing compound is selected from avobenzone, octylmethoxycinnamate and combinations thereof.

84. The method of claim 82, wherein the UV-A protection provided is evaluated using a method chosen from the FDA Star Method, the COLIPA Guidelines, the Boots Method, and the Diffey Protocol.

85. A crosslinked UV absorbing complex polyol polyester polymer that is the reaction product of a monofunctional agent comprising an UV absorbing moiety that has a structure represented by (XIII): 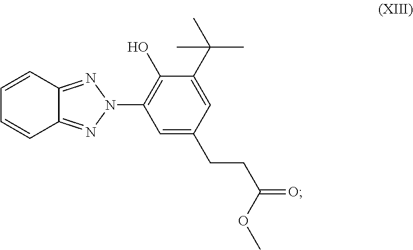
and additional reagents comprising those having the structures represented by (XIV) to (XV):

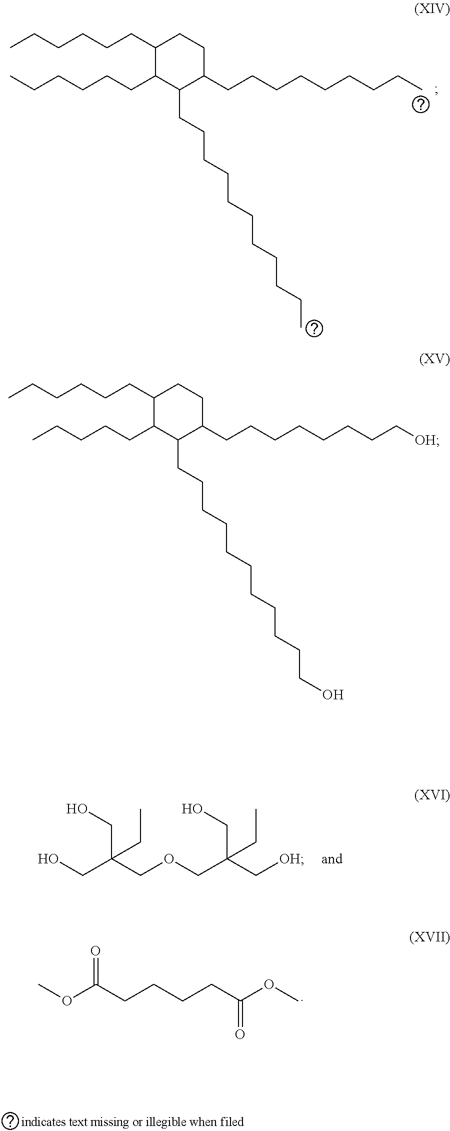

86. A personal care composition comprising the polymer of claim 85.

87. The composition of claim 86, further comprising one or more components selected from a vegetable oil, a surfactant, a lipid, an alcohol, a wax, a pigments, a vitamin, a fragrance, a bleaching agent, an antibacterial agent, an anti-inflammatory agent, an antimycotic agent, a thickener, a gum, a starch, a chitosan, a polymeric material, a cellulosic material, a glycerin, a protein, an amino acid, a keratin fiber, a fatty acid, a siloxane, a botanical extract, an abrasive, a chemical exfoliant, a mechanical exfoliant, an anticaking agent, an antioxidant agent, a binder, a clay, a biological additive, a buffering agent, a bulking agent, a chelating agent, a film former, an humectant, an opacifying agent, a pH adjuster, a preservative, a propellant, a reducing agent, a skin darkening agent, an essential oils, a skin sensates, and combinations of these.

88. The composition of claim 86, further comprising an further comprising an optical brightener.

89. The composition of claim 88, wherein the optical brightener is represented by the formula (XII): 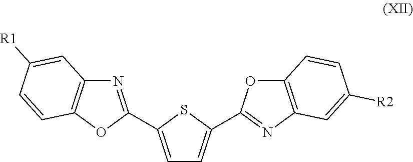
in which R¹ and R² are independently chosen from branched or unbranched, saturated or unsaturated alkyl radicals having 1 to 10 carbon atoms.

90. A method of increasing the photostability of a personal care composition comprising incorporating the UV absorbing complex polyol polyester polymer of claim 85 into a personal care composition.

91. A method of protecting a portion of the skin, hair and or nails of a mammal from damage by UV light comprising applying an effective amount of the UV absorbing complex polyol polyester polymer of claim 85 to the portion of skin, hair or nails.

92. A method of photostabilizing a photoprotective personal care composition that contains a non-polymeric UV absorbing compound comprising incorporating into the composition an effective amount of the polymer of claim 85.

93. The method of claim 92, wherein the non-polymeric UV absorbing compound is selected from avobenzone, octylmethoxycinnamate and combinations thereof.

94. A method of increasing the UV-A/UV-B ratio of a composition that contains a non-polymeric UV absorbing compound comprising incorporating into the composition an effective amount of the polymer of claim 92, wherein the increase in protection is evaluated using the Boots Method.

95. A method of increasing the Sun Protection Factor of a photoprotective personal care composition that contains a non-polymeric UV absorbing compound comprising incorporating into the composition an effective amount of the polymer of claim 92.

96. The method of claim 95, wherein the non-polymeric UV absorbing compound is selected from avobenzone, octylmethoxycinnamate and combinations thereof.

97. A method of increasing the UV-A protection provided by a photoprotective personal care composition that contains a non-polymeric UV absorbing compound comprising incorporating into the composition an effective amount of the polymer of claim 85.

98. The method of claim 85, wherein the UV-A protection provided is evaluated using a method chosen from the FDA Star Method, the COLIPA Guidelines, the Boots Method, and the Diffey Protocol.

99. A optical brightener represented by the formula (XII): 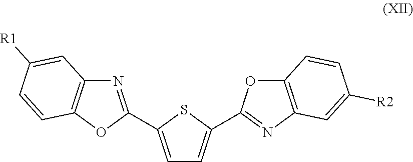
in which R¹ and R² are independently chosen from branched or unbranched, saturated or unsaturated alkyl radicals having 1 to 10 carbon atoms.

100. A personal care composition containing the optical brightener of claim 99.

101. A personal care composition comprising an optical brightener represented by the formula (XII): 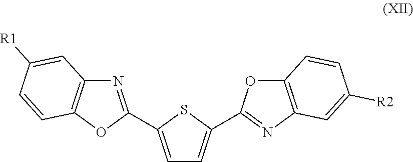
in which and R² are independently chosen from branched or unbranched, saturated or unsaturated alkyl radicals having 1 to 10 carbon atoms; and at least one of a UV absorbing complex polyester polymer selected from polymer A, polymer B, polymer C, polymer D, polymer E, and polymer F,
wherein polymer A is the product of a reaction scheme comprising:
(i) the esterification of a polyol and a dianhydride, wherein the esterification is carried out under conditions that facilitate substantially only anhydride opening, to form a polyester polymer comprising at least two pendant carboxylic groups, and at least two hydroxyl groups; and
(ii) the reaction of at least one pendant carboxylic group and at least one terminal hydroxyl group of the polyester polymer with an epoxide having a functional group, wherein the epoxide comprises an UV absorbing moiety;
wherein polymer B is a reaction product of the esterification of a diol, and an UV absorbing dianhydride that benzophenone moiety to yield a polyester polymer that comprises a pendent carboxylic acid and a terminal hydroxyl group as represented by Formula (IX): 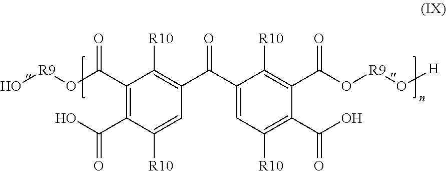
wherein R⁹ is independently selected from a hydrocarbon group having 2 to 54 carbon atoms, and 0 to 30 ether linkages, R¹⁰ is independently —H, or —OH, and n is an integer of 1 to 1000, and the subsequent etherification of the functional group of the expoxide with the hydroxyl and/or carboxylic acid groups of the polyester polymer. the polyester polymer;
wherein polymer C is a reaction product of a esterification of a diol and a dianhydride that is not UV absorbing, yielding a polyester polymer that comprises at least two pendant carboxylic acid groups and two terminal hydroxyl groups represented by Formula (X) 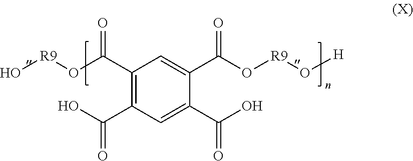
wherein R⁹ is independently selected from a hydrocarbon group having 2 to 54 carbon atoms, and 0 to 30 ether linkages, and n is an integer of 1 to 1000 and the etherification of the functional group of the expoxide with at least one of the hydroxyl and/or carboxylic acid groups of the polyester polymer;
wherein polymer D is A linear UV absorbing complex polyol polyester polymer represented by Formula (XI): 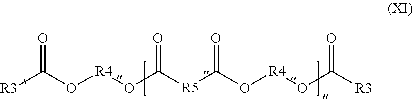
wherein R³ is independently selected from an UV absorbing moiety; R⁴ and R⁵ are each independently selected from a hydrocarbon group, and n is an integer of 1 to 1000;
wherein polymer E is a crosslinked UV absorbing complex polyol polyester polymer that is reaction product of a random copolyesterification esterification reaction and/or the esterification product of: a monofunctional carboxylic acid and/or ester that comprises an UV absorbing moiety, at least one of a diol, a polyol, a diacid and/or an ester, wherein the polymer has an UV absorbing functionality of greater than 2.0; and
wherein polymer F is a crosslinked UV absorbing complex polyol polyester polymer that is the reaction product of a monofunctional agent comprising an UV absorbing moiety that has a structure represented by (XIII): 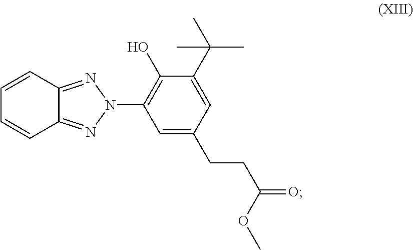
and additional reagents comprising those having the structures represented by (XIV) to (XV): [chemical structure]
